SEQUENCE LISTING (80 sequences)

SEQ ID NO: 1 - protein, 15 aa, Artificial Sequence
  Description of Artificial Sequence Synthetic peptide
SGYSSPGSPGTPGSR

SEQ ID NO: 2 - protein, 12 aa, Artificial Sequence
  Description of Artificial Sequence Synthetic peptide
KSYGXEXTLXAE

SEQ ID NO: 3 - protein, 12 aa, Artificial Sequence
  Description of Artificial Sequence Synthetic peptide
VDAAVTPEERHC

SEQ ID NO: 4 - protein, 18 aa, Artificial Sequence
  Description of Artificial Sequence Synthetic peptide
QEFEVMEDHAGTYGLGDR

SEQ ID NO: 5 - protein, 20 aa, Artificial Sequence
  Description of Artificial Sequence Synthetic peptide
DQGGYTMHQDQEGDTDAGLK

SEQ ID NO: 6 - protein, 21 aa, Artificial Sequence
  Description of Artificial Sequence Synthetic peptide
KDQGGYTMHQDQEGDTDAGLK

SEQ ID NO: 7 - protein, 15 aa, Artificial Sequence
  Description of Artificial Sequence Synthetic peptide
SGYSSPGSPGTPGSR

SEQ ID NO: 8 - protein, 8 aa, Homo sapiens
TPGTPGTP

SEQ ID NO: 9 - protein, 8 aa, Artificial Sequence
  Description of Artificial Sequence Synthetic peptide
XPGXPGTP

SEQ ID NO: 10 - protein, 8 aa, Homo sapiens
SPGSPGTP

SEQ ID NO: 11 - protein, 8 aa, Homo sapiens
SPSASKSR

SEQ ID NO: 12 - protein, 10 aa, Homo sapiens
TPPKSPATPK

SEQ ID NO: 13 - protein, 13 aa, Artificial Sequence
  Description of Artificial Sequence Synthetic peptide
KRREILSRRPSYR

SEQ ID NO: 14 - protein, 12 aa, Artificial Sequence
  Description of Artificial Sequence Synthetic peptide
VDAAVTPEERHC

SEQ ID NO: 15 - protein, 9 aa, Homo sapiens
IETHKLTFR

SEQ ID NO: 16 - protein, 7 aa, Homo sapiens
EPSTVSR

SEQ ID NO: 17 - protein, 17 aa, Homo sapiens
KTTAAGGESALAPSVFK

SEQ ID NO: 18 - protein, 19 aa, Homo sapiens
TTAAFFESALAPSVFKQAK

SEQ ID NO: 19 - protein, 7 aa, Homo sapiens
VSDGVTK

SEQ ID NO: 20 - protein, 7 aa, Homo sapiens
RGVSGDR

SEQ ID NO: 21 - protein, 8 aa, Homo sapiens
LNFREHAK

SEQ ID NO: 22 - protein, 5 aa, Homo sapiens
MADPR

SEQ ID NO: 23 - protein, 7 aa, Homo sapiens
GTSNATR

SEQ ID NO: 24 - protein, 10 aa, Homo sapiens
TPPSSGEPPK

SEQ ID NO: 25 - protein, 14 aa, Homo sapiens
TPPSSGEPPKSGDR

SEQ ID NO: 26 - protein, 19 aa, Homo sapiens
SGDRSGYSSPGSPGTPGSR

SEQ ID NO: 27 - protein, 19 aa, Homo sapiens
SGDRSGYSSPGSPGTPGSR

SEQ ID NO: 28 - protein, 17 aa, Homo sapiens
SGYSSPGSPGTPGSRSR

SEQ ID NO: 29 - protein, 12 aa, Homo sapiens
SRTPSLPTPPTR

SEQ ID NO: 30 - protein, 8 aa, Homo sapiens
SPSSAKSR

SEQ ID NO: 31 - protein, 11 aa, Homo sapiens
SPVVSGDTSPR

SEQ ID NO: 32 - protein, 7 aa, Homo sapiens
VSDGVTK

SEQ ID NO: 33 - protein, 11 aa, Homo sapiens
VSDGVTKSPEK

SEQ ID NO: 34 - protein, 7 aa, Homo sapiens
RGVSGDR

SEQ ID NO: 35 - protein, 12 aa, Homo sapiens
TPGTPGTPSYPR

SEQ ID NO: 36 - protein, 16 aa, Homo sapiens
ARVDHGAEIITQSPGR

SEQ ID NO: 37 - protein, 12 aa, Artificial Sequence
  Description of Artificial Sequence Synthetic peptide
VDAAVTTEERHC

SEQ ID NO: 38 - protein, 22 aa, Mus musculus
LSEEACPGVLSVAPTVTQPPGR

SEQ ID NO: 39 - protein, 27 aa, Mus musculus
AVCLAAQSIAMGDSTIVVAGGMENMSK

SEQ ID NO: 40 - protein, 20 aa, Mus musculus
IAALAGCKTTTSAAMVHCLR

SEQ ID NO: 41 - protein, 17 aa, Mus musculus
LSDGSECSSGICCNSCK

SEQ ID NO: 42 - protein, 15 aa, Mus musculus
GTVLRYSGSSAALER

SEQ ID NO: 43 - protein, 19 aa, Mus musculus
VVDGTLCTPDSTSVCVQGK

SEQ ID NO: 44 - protein, 15 aa, Mus musculus
WSKCSVTCGIGIMER

SEQ ID NO: 45 - protein, 20 aa, Mus musculus
CGVCGGDNSSCTKIIGTFNK

SEQ ID NO: 46 - protein, 19 aa, Mus musculus
ESTTSEQSARMTAMDNASK

SEQ ID NO: 47 - protein, 14 aa, Mus musculus
SSSSSASPSSPSSR

SEQ ID NO: 48 - protein, 20 aa, Mus musculus
EPCGGTGSTGAGGGTNGGVR

SEQ ID NO: 49 - protein, 8 aa, Mus musculus
LSSGMPAR

SEQ ID NO: 50 - protein, 18 aa, Mus musculus
CSLFTCPQNETLFNSRTR

SEQ ID NO: 51 - protein, 22 aa, Mus musculus
GHSTSTDDTSDPTSKEFEALIK

SEQ ID NO: 52 - protein, 22 aa, Mus musculus
SSGAGGGGSGGSGAGGLIGLMK

SEQ ID NO: 53 - protein, 10 aa, Mus musculus
SSPSIICMPK

SEQ ID NO: 54 - protein, 8 aa, Mus musculus
GLDSTTPR

SEQ ID NO: 55 - protein, 17 aa, Mus musculus
VSSVASSALVSSLHMMK

SEQ ID NO: 56 - protein, 21 aa, Mus musculus
TKAPGPGPEGSSSLHPNPTDK

SEQ ID NO: 57 - protein, 27 aa, Mus musculus
NEGEAAASTGGPPSGSSASTTSSSSAR

SEQ ID NO: 58 - protein, 25 aa, Mus musculus
GPGGGAMVSVGGATFMVGDSGYMTL

SEQ ID NO: 59 - protein, 9 aa, Mus musculus
GSSSASVTK

SEQ ID NO: 60 - protein, 21 aa, Mus musculus
SSPNPFVGSPPKGLSVPNGVK

SEQ ID NO: 61 - protein, 25 aa, Mus musculus
SMNPNVSMVSSASSSPSSSRTPQAK

SEQ ID NO: 62 - protein, 15 aa, Mus musculus
DCKSFPGGAGPLGCK

SEQ ID NO: 63 - protein, 15 aa, Mus musculus
CRGSSGAGGPATCSR

SEQ ID NO: 64 - protein, 20 aa, Mus musculus
AGTLDNIYATLVGTCGESPK

SEQ ID NO: 65 - protein, 16 aa, Mus musculus
GDVGPGESGGPLTPRR

SEQ ID NO: 66 - protein, 15 aa, Mus musculus
AWAAAAANSTVWSDK

SEQ ID NO: 67 - protein, 19 aa, Mus musculus
ESITSAVASLPGSPAPSPR

SEQ ID NO: 68 - protein, 18 aa, Mus musculus
STPVSPPLLLPATGGTSK

SEQ ID NO: 69 - protein, 11 aa, Mus musculus
GECGSGGGDSK

SEQ ID NO: 70 - protein, 11 aa, Mus musculus
SSGASVSALQR

SEQ ID NO: 71 - protein, 14 aa, Mus musculus
GCTLSAEERAALER

SEQ ID NO: 72 - protein, 21 aa, Mus musculus
CQDSPEGGSSGPGESSEERNK

SEQ ID NO: 73 - protein, 19 aa, Mus musculus
NQGGYGGSSSSSSYGSGRR

SEQ ID NO: 74 - protein, 18 aa, Mus musculus
NQGGYGGSSSSSSYGSGR

SEQ ID NO: 75 - protein, 13 aa, Mus musculus
LSQTMLGHVGLNK

SEQ ID NO: 76 - protein, 15 aa, Mus musculus
SFPSSPSKGGACPSR

SEQ ID NO: 77 - protein, 19 aa, Mus musculus
AGGGGAGGAGGGSGGGGSR

SEQ ID NO: 78 - protein, 397 aa, Homo sapiens
FKTGRGRISTPERKVAKKEPSTVSRDEVRRKKAVYKKAELAKKTEVQAHSPSRKFILKPAIKYTRPTHLSCVKRKTTAAGGESALAPSVFKQAKDKVSDGVTKSPEKRSSLPRPSSILPPRRGVSGDRDENSFSLNSSISSSARRTTRSEPIRRAGKSGTSTPTTPGSTAITPGTPPSYSSRTPGTPGTPSYPRTPHTPGTPKSAILVPSEKKVAIIRTPPKSPATPKQLRLINQPLPDLKNVKSKIGSTDNIKYQPKGGQVQIVTKKIDLSHVTSKCGSLKNIGHRPGGGRVKIESVKLDFKEKAQAKVGSLDNAHHVPGGGNVKIDSQKLNFREHAKARVDHGAEIITQSPGRSSVASPRRLSNVSSSGSINLLESPQLATLAEDVTAALAKQGL

SEQ ID NO: 79 - protein, 431 aa, Homo sapiens
MEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 80 - protein, 9 aa, Artificial Sequence
  Description of Artificial Sequence Synthetic peptide
LSRRPSYRK

(19) United States
(12) Patent Application Publication
(10) Pub. No.: US 2005/0261475 A1
(43) Pub. Date: Nov. 24, 2005

(54) Solid-phase capture-release-tag methods for phosphoproteomic analyses

(75) Inventors: Huang-Chun Tseng, Medford, MA (US); Li-Huei Tsai, Cambridge, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Harvard Medical School, Boston, MA (US)

(21) Appl. No.: 11/058,735

(22) Filed: Feb. 14, 2005

Related U.S. Application Data
(60) Provisional application No. 60/544,748, filed on Feb. 13, 2004.

(51) Int. Cl. C07K 14/47; C07K 1/02

(52) U.S. Cl. 530/333; 530/352

(57) ABSTRACT

One aspect of the present invention relates to a method of labeling a peptide or protein comprising a phosphorylated amino acid comprising: a) removing from the peptide or protein the phosphate group via a β-elimination to form a double bond conjugated to a C═O group; b) reacting the double bond from step a) with a nucleophile comprising a tag on a solid support; and c) removing the solid support from the product of step b) forming the labeled peptide or protein. A second aspect of the invention relates to a solid support comprising a linker and tag between the solid support and a nucleophile, wherein the tag comprises a label.

### RELATED APPLICATIONS

This application claims the benefit of priority to U.S. Provisional Patent Application Ser. No. 60/544,748, filed Feb. 13, 2004; the contents of which is incorporated by reference.

### BACKGROUND OF THE INVENTION

In the post-genomic era, functional proteomics will accelerate the understanding of complicated biological pathways regulated by enzymatic activity. Pandey, A. & Mann, M. Proteomics to study genes and genomes. Nature 405, 837-846 (2000). Protein phosphorylation/dephosphorylation catalyzed by protein kinases/phosphatases is essential in a variety of signaling processes. Edelman, A. M., Blumenthal, D. K. & Krebs, E. G. Protein serine/threonine kinases. Annu Rev Biochem 56, 567-613 (1987); Hunter, T. Protein kinases and phosphatases: the yin and yang of protein phosphorylation and signaling. Cell 80, 225-236 (1995). Analysis of complex phosphorylation events has the potential to reveal mechanisms regulating phosphorylated proteins. However, current research for determining the phosphorylation events are still focused on method improvement. Mann, M. et al. Analysis of protein phosphorylation using mass spectrometry: deciphering the phosphoproteome. Trends Biotechnol 20, 261-268 (2002).

Beta-elimination of phosphates on serine/threonine amino acids is increasingly utilized for phosphorylation analysis. Under alkaline conditions, phosphate moieties on the serine and threonine residues undergo β-elimination. The resulting dehydroanaline/dehydrobutyric acid subsequently servers as a Michael acceptors. Meyer, H. E., Hoffmann-Posorske, E. & Heilmeyer, L. M., Jr. Determination and location of phosphoserine in proteins and peptides by conversion to Sethylcysteine. Methods Enzymol 201, 169-185 (1991); Oda, Y., Nagasu, T. & Chait, B. T. Enrichment analysis of phosphorylated proteins as a tool for probing the phosphoproteome. Nat Biotechnol 19, 379-382 (2001). Previous studies have shown that sulphur nucleophiles can form covalent thioether bonds at phospho-serine/threonine residues during Michael addition. Meyer, H. E., Hoffmann-Posorske, E. & Heilmeyer, L. M., Jr. Determination and location of phosphoserine in proteins and peptides by conversion to Sethylcysteine. Methods Enzymol 201, 169-185 (1991); Annan, W. D., Manson, W. & Nimmo, J. A. The identification of phosphoseryl residues during the determination amino acid sequence in phosphoproteins. Anal Biochem 121, 62-68 (1982). Recently, Oda et al. applied a liquid-phase (β-elimination-based reaction followed by a NeutrAvidin bead purification to enrich phospho-peptides from yeast. Oda, Y., Nagasu, T. & Chait, B. T. Enrichment analysis of phosphorylated proteins as a tool for probing the phosphoproteome. Nat Biotechnol 19, 379-382 (2001). Surprisingly, only one phospho-peptide was obtained from this yeast phospho-proteome study, raising a concern of feasibility for the use of β-elimination/Michael addition for in vivo phospho-proteomic studies. Oda, Y., Nagasu, T. & Chait, B. T. Enrichment analysis of phosphorylated proteins as a tool for probing the phosphoproteome. Nat Biotechnol 19, 379-382 (2001). In another study, Knight et al. designed a phosphorylation-specific proteolysis application using the elimination methodology to yield lysine-like residues at phosphorylation sites. Knight, Z. A. et al. Phosphospecific proteolysis for mapping sites of protein phosphorylation. Nat Biotechnol 21, 1047-1054 (2003). Whether this method is practical for complex cellular samples remains unclear. The present invention takes advantage of the ease of chemical diversification and the large reactive surface area of water-swellable beads commonly used in solid-phase peptide synthesis to design a novel method which combines β-elimination with solid-phase Michael addition in a convenient one-pot procedure.

### SUMMARY OF THE INVENTION

In part, the present invention relates to a method of labeling a peptide or protein comprising a phosphorylated amino acid comprising: a) removing from the peptide or protein the phosphate group via a β-elimination to form a double bond conjugated to a C═O group; b) reacting the double bond from step a) with a nucleophile comprising a tag and a solid support; and c) removing the solid support from the product of step b) forming the labeled peptide or protein.

In a further embodiment, the removing of the phosphate group via a β-elimination is base catalyzed. In a further embodiment, the removing of the phosphate group via a β-elimination is catalyzed by an amine-containing base, a hydroxide-containing base, or mixtures thereof. In a further embodiment, the removing of the phosphate group via a β-elimination is catalyzed by a hydroxide-containing base. In a further embodiment, the removing of the phosphate group via a β-elimination is catalyzed by Ba(OH)₂.

In a further embodiment, the nucleophile is selected from the group consisting of oxygen-containing nucleophiles, nitrogen-containing nucleophiles, and sulfur-containing nucleophiles. In a further embodiment, the nucleophile comprises a sulfur atom. In a further embodiment, one or more other potential nucleophiles within the peptide or protein are protected before step a). In a further embodiment, one or more other potential nucleophiles within the peptide or protein are protected before step a) via acylation or alkylation.

In a further embodiment, the tag comprises a deuterium ion. In a further embodiment, the tag comprises eight deuterium ions.

In a further embodiment, the solid support is a glass, derivatized glass, silicon, plastic or a resin. In a further embodiment, the solid support is a bead. In a further embodiment, the solid support is a resin bead. In a further embodiment, the solid support is a polystyrene/poly(ethylene glycol)resin bead.

In a further embodiment, the solid support is removed by acid hydrolysis. In a further embodiment, the efficiency in detecting the peptides and proteins is increased by about 60% as compared to detection by mass spectrometry.

In a preferred embodiment, there is a linker between said solid support and said nucleophile-containing tag. In a further embodiment, the linker is a NH₂-functionalized RINK linker (NH₂-RINK linker), 4-hydroxymethyl-phenylacetamidomethyl linker (PAM linker), 4-(1′,1′-dimethyl-1′-hydroxypropyl)phenoxyacetyl linker (DHPP linker), 4-hydroxymethylphenoxy linker (Wang linker), super Acid Sensitive linker (SASRIN linker), hypersensitive acid-labile tris(alkoxy)benzyl ester linker (HAL linker), 4-(2′,4′-dimethoxyphenyl-Fmoc-aminomethyl)-phenoxy linker (RINK linker), N-[9-(Hydroxymethyl)-2-fluorenyl]succinamic acid linker (HMFS linker), fmoc-(aminomethyl)-3,5-dimethoxyvaleric acid linker (Pal linker), 9-xanthenyl linker (XAN linker) or safety-catch acid-labile linker (SCAL linker). In a further embodiment, the linker is a NH₂-functionalized RINK linker (NH₂-RINK linker) or 4-(2′,4′-dimethoxyphenyl-Fmoc-aminomethyl)-phenoxy linker (RINK linker). In a further embodiment, the linker is a NH₂-functionalized RINK linker (NH₂-RINK linker).

In a preferred embodiment, the solid support is a polystyrene/poly(ethylene glycol) resin bead; the linker is a NH₂-functionalized RINK linker (NH₂-RINK linker); the tag comprises a deuterium ion; and the nucleophile comprises a sulfur atom. In a further embodiment, the solid support is a polystyrene/poly(ethylene glycol) resin bead; the linker is a NH2-functionalized RINK linker (NH2-RINK linker); the tag and nucleophile is —C(═O)CD₂CD₂CD₂CD₂CH₂NH—C(═O)CH₂SX; and X is a cation.

In another embodiment, the present invention relates to a solid support comprising a linker, a tag and a nucleophile.

In a further embodiment, wherein the solid support is a glass, derivatized glass, silicon, plastic or a resin. In a further embodiment, the solid support is a bead. In a further embodiment, wherein the solid support is a resin bead. In a further embodiment, wherein the solid support is a polystyrene/poly(ethylene glycol) resin bead.

In a further embodiment, wherein the tag contains a deuterium label. In a further embodiment, the tag comprises eight deuterium ions.

In a further embodiment, wherein the linker comprises an amide moiety. In a further embodiment, wherein the linker comprises an aryl moiety. In a further embodiment, wherein the linker is a NH₂-functionalized RINK linker (NH₂-INK linker), 4-hydroxymethyl-phenylacetamidomethyl linker (PAM linker), 4-(1′,1′-dimethyl-1′-hydroxypropyl)phenoxyacetyl linker (DHPP linker), 4-hydroxymethylphenoxy linker (Wang linker), super Acid Sensitive linker (SASRIN linker), hypersensitive acid-labile tris(alkoxy)benzyl ester linker (HAL linker), 4-(2′,4′-dimethoxyphenyl-Fmoc-aminomethyl)-phenoxy linker (RINK linker), N-[9-(Hydroxymethyl)-2-fluorenyl]succinamic acid linker (HMFS linker), fmoc-(aminomethyl)-3,5-dimethoxyvaleric acid linker (Pal linker), 9-xanthenyl linker (XAN linker) or safety-catch acid-labile linker (SCAL linker). In a further embodiment, wherein the linker is a NH₂-functionalized RINK linker (NH₂-RINK linker) or 4-(2′,4′-dimethoxyphenyl-Fmoc-aminomethyl)-phenoxy linker (RINK linker). In a further embodiment, wherein the linker is a NH₂-functionalized RINK linker (NH₂-RINK linker).

In a further embodiment, wherein the nucleophile is selected from the group consisting of oxygen-containing nucleophiles, nitrogen-containing nucleophiles, and sulfur-containing nucleophiles. In a further embodiment, wherein the nucleophile comprises a sulfur atom.

In a preferred embodiment, the solid support is a polystyrene/poly(ethylene glycol) resin bead; the linker is a NH₂-functionalized RINK linker (NH₂-RINK linker); the tag comprises a deuterium ion; and the nucleophile comprises a sulfur atom. In a further embodiment, the solid support is a polystyrene/poly(ethylene glycol) resin bead; the linker is a NH₂-functionalized RINK linker (NH₂-RINK linker); the tag and nucleophile is —C(═O)CD₂CD₂CD₂CD₂CH₂NH—C(═O)CH₂SX; and X is a cation.

### BRIEF DESCRIPTION OF DRAWINGS

FIG. 1 depicts A. scheme of solid-phase Michael addition methodology. The method involves a sample preparation step (1. alkylation), a capturing step (2. (β-elimination and 3. Machiel addition), a washing step (between 3 and 4), and a releasing-tagging step (4. acid cleavage). Recovered peptides are analyzed by tandem mass spectrometry. B. Detailed chemical structure of the nucleophile resin. 6-(2-mercapto-acetylamine)-hexanoic acid (DO) RINK resin and it's deuterium-coded (D8) version. Eight deuterium atoms were positioned in the hexanoic acid spacer in the D8 version. The thiol group serves as a nucleophile in the subsequent Michael addition step. The tag is indicated with an underline.

FIG. 2 depicts MALDI-TOF mass spectrometric analysis of synthetic phospho-peptides before and after the method. A. The spectrum of phospho-CREB peptide before SMA. B. The spectrum of phospho-CREB peptide after the method treatment using the DO resin. C. The spectrum of phospho-CREB peptide after the method treatment using the D8 resin. The identities of ions are indicated with arrows. Metastable peaks are indicated with arrow heads. The MALDI-TOF analysis of phospho-Thr APP peptides before and after the method are shown in D and E, respectively.

FIG. 3 depicts the summary graphs of MS-based phosphorylation determination on mouse brain synaptosomal membrane proteins. Three MS-based methods are used: conventional, neutral-loss of phosphoric acid, and solid-phase chemical tag determination. A. Percentage of phospho-peptides in total peptides. B. Percentage of phospho-proteins in top 50 proteins. C. percentage of phospho-(S/T)P containing peptide in total phosphopeptides. The original numbers are shown at their corresponding groups.

FIG. 4 depicts A Immunoblotting assay of Tau proteins. Tau-5 antibody recognizes all forms of Tau proteins. AT-8 antibody recognizes Ser202/Thr205 phosphorylated Tau. PHF-1 is a phosphorylation-specific antibody to phospho-Ser396. Thr231 antibody recognizes Thr231 phosphorylation. B. Comparison of Cdk5 phosphorylation of MAP2 and Tau. Partial sequences of MAP2 (NCBI access number NP 002365) and Tau (access number QRHUTl) are shown. Pairwise alignment of MAP2 and Tau is done in BioEdit using PAM 120 algorithm. Black blocks indicate identical amino acids between proteins; gray blocks for amino acids with similar chemical and physical properties. Dashes represent probable gaps. Box indicates the regions of microtubule-binding repeats. Phosphorylated residues are indicated with stars. Note that most of the similar phosphorylations between the two proteins appear in highly homologous regions.

FIG. 5 depicts the scope of proteins in an era of proteomics and functional proteomics.

FIG. 6 depicts the summary of Q-TOF MS/MS Spectrum of the m/z 634.8 ([MH₃]³⁺) ions.

FIG. 7 depicts the protocol of Tau phosphorylation determination using SMA MS.

FIG. 8 depicts phosphorylation sites of cellular Tau and MAP2.

FIG. 9A. Scheme of solid-phase Michael addition methodology. The method involves a sample preparation step (1. alkylation), a capturing step (2. β-elimination and 3. Michael addition), a washing step (between 3 and 4), and a tag-releasing step (4. acid-mediated cleavage). Recovered peptides are analyzed by mass spectrometry. B. Detailed chemical structure of the nucleophile resin. 6-(2-mercapto-acetylamine)-hexanoic acid RINK resin. C. MALDI-TOF mass spectra of 1 pmol phospho-CREB peptides (1) before SMA treatment and (2) after SMA treatment. Metastable ions are indicated by arrow heads.

FIG. 10 depicts MALDI-TOF mass spectrometric analysis of phosphorylated microtubule-associated protein Tau. Recombinant Tau (100 pmol) was phosphorylated by active CDK5 in vitro. Tryptic Tau peptides prepared from samples in the absence and presence of active CDK5 are shown in A and B, respectively. As a control, CDK5-phosphorylated peptides were treated with alkaline phosphatases. The MS spectrum of dephosphorylated peptides is shown in C. One phospho-peptide, SGYSSPGSPGTPGSR plus 1 phosphate, was detected and is indicated by an arrow. The non-phospho-peptide counterpart is indicated by an arrow head. The phosphorylated and dephosphorylated samples were prepared by SMA prior to MS analysis (D and E, respectively). The peptide with the tag, representing the phospho-peptide, is detected in spectrum D, but not in E. Some unexpected molecular ions also appeared in the MALDI-TOF MS spectra. These molecular ions do not match any Tau peptides containing the tag moiety. Their molecular masses can not be explained by reaction of SMA with any unphosphorylated Tau tryptic peptides. The phospho-peptide was also isolated by using IMAC, another technique for enriching phospho-peptides (F).

FIG. 11 depicts results of analysis of Tau phosphorylation by CDK5. A. Tau antibodies are used to detect tau phosphorylation. Tau-5 is an antibody recognizing total Tau. AT-8 antibody recognizes phosphorylation of Ser202/Thr205 of Tau. pThr231 antibody recognizes phosphorylation of Tau at Thr231. B. The MS/MS mass spectrum of the parent ion m/z 790.35 (2+) acquired from an ion-trap instrument. Sequence determination is shown above. Major detected daughter ions are indicated in the spectrum.

FIG. 12 depicts sensitivity and specificity analysis for the SMA. Phospho-CREB peptide (1 nmol) was incubated with sequence: LSRRP(phospho-S)YRK (as shown in A) in a β-elimination solution containing 40 mM BaOH, 20% methanol at 50° C., 30 minutes to yield dehydro-CREB peptides (shown in B). All phospho-CREB peptides were converted to their dehydro-forms. After the dehydro-peptides were incubated with the SMA resin at 50° C. for one hour, all of 1 nmol of the dehydro-peptides can be captured by 50 μL (bed volume in DNF solvent) of the resin. No dehydropeptide was detected in unbound fraction, as judged by a MALDI-TOF analysis shown in C. Phospho-CREB peptide (1 pmol) was mixed with 10 μg of non-phosphoryl peptide library with KSYGXEXTLXAE sequences (X=any amino acids except cysteine, 6859 different peptides in total). The MALDI-TOF spectrum of the mixture is shown in D. The weight ratio of the phospho-peptide to non-phosphoryl peptides is about 1:8000. After the SMA treatment at 50° C. on the mixture, the tagged-CREB peptide was recovered. Occasionally, the dehydro-form of the peptide is detected. Spectrum F is the result when SMA resin was incubated with the non-phosphoryl peptide library. This result suggests that the SMA resin has little reactivity with non-phosphoryl peptides.

### DETAILED DESCRIPTION OF THE INVENTION

Overview

Remarkably, we have discovered a chemistry-based capture-release-tag method for the isolation of, e.g., complex phospho-Ser/Thr-containing peptides by liquid β-elimination combined with a solid-phase Michael addition. For example, the free thiol group of a 6-(mercapto-acetylamino)-hexanoic acid functionalized resin may be used as an immobilized Michael donor to capture dehydro-serine/threonine peptides. After trifluoracetic acid-mediated release of captured peptides from the resin, peptides are labeled with a 6-(2-mercapto-acetylamine)-hexanoic amide tag at phosphorylated sites. In this embodiment, the method enables the analysis of the phosphorylation status of recombinant and cellular microtubule-associated protein Tau in the presence of active Cyclin-dependent kinase 5 (CDK5). In addition, a more global analysis of phosphorylation was carried out with the cellular heat stable fractions to enrich for microtubule associated proteins. Among the recovered phospho-peptides, we found that a novel CDK5 substrate microtubule-associated protein 2 (MAP-2) is phosphorylated on residues that are within a homologous region of Tau.

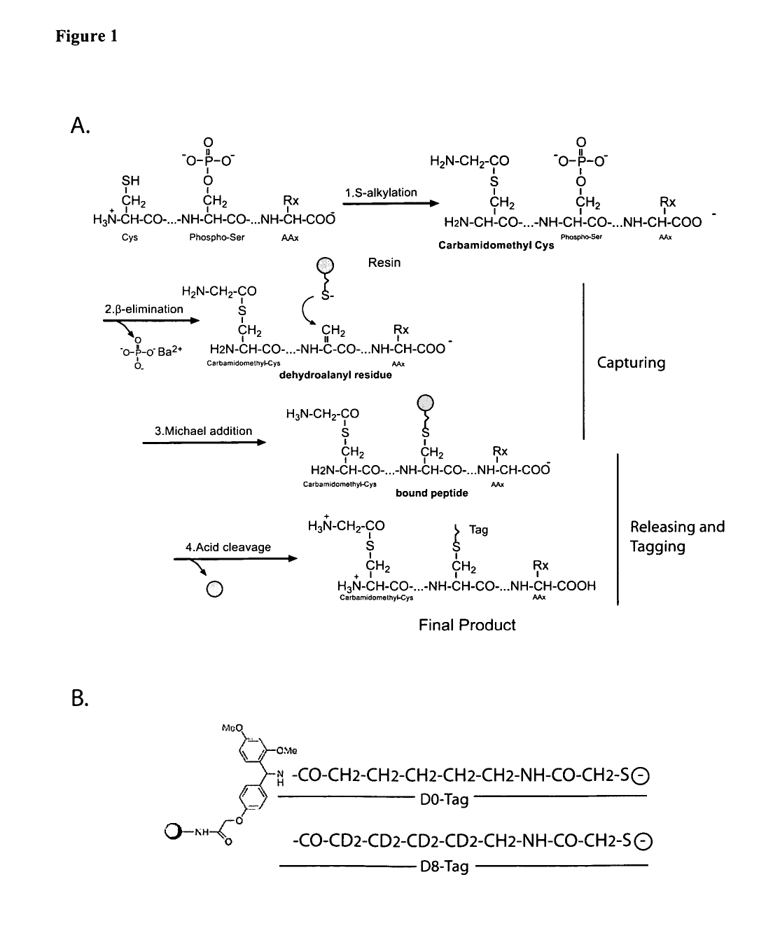

In one embodiment, the present invention discloses solid state material that captures phospho-peptides through beta-elimination/Michael addition reactions. This material introduces the capture-label (or tag)-release concept to functional proteomic tool development and successfully demonstrates its practical application on proteomic research. The approach disclosed herein is illustrated in FIG. 1A. Cysteine residues of tryptic phospho-peptides are reduced and alkylated. The core chemical reactions, β-elimination and solid-phase Michael addition, occur under alkaline reaction conditions. Phosphoserine and phospho-threonine are converted to dehydroanaline and dehydrobutyric acid, respectively. Newly formed double bonds between the α and β carbons of dehydroanaline/dehydrobutyric acid subsequently serve as Michael acceptors to which nucleophiles react. Unreacted peptides and debris are then washed away. Acid-mediated cleavage releases captured and purified peptides in tagged form. Phosphorylation sites on released products are then determined and quantified by tandem mass spectrometry. The chemical structure of the nucleophilic group used in this report is depicted in FIG. 1B.

Remarkably, we have used a solid-phase Michael addition for the purpose of phospho-peptide enrichment. Not only have we demonstrated mass spectrometry-based phosphorylation determination, but we have also extended the application of solid-phase chemistry to biological samples. The thio-nucleophilic resin fulfills two functions: the capturing and tagging of phospho-peptides in one single reaction vessel when β-elimination and Michael reaction occur. The strategy of replacing labile phosphates with a more stable chemical tag allows increased detection sensitivity in mass spectrometry. Moreover, the concept of solid-phase chemistry can be extended to other labeling applications. For example, O-linked glycosylation performs β-elimination under, e.g., sodium hydroxide conditions. Therefore, the resin may be used to enrich for glycosylated peptides derived from lectin-purified samples. Using the microtubule associated proteins Tau and MAP2 as examples, we have shown that SMA allows identification of phosphorylation sites of proteins present in complex whole cell extracts. The finding of a potential CDK5 motif on both microtubule-associated proteins and the presence of similar phosphorylation events in mouse MAP2 and Tau suggests that phosphorylation of MAP2 and Tau may be regulated by kinases in a similar fashion.

Solid Supports

As disclosed herein, the solid support can be a poly-styrene resin. In a preferred embodiment, the resin is an ArgoGel® resin (see Exemplification). ArgoGel® resins are solid supports that consist of a poly(ethylene glycol) (PEG) grafted onto 1% cross-linked polystyrene with the linkers (x) attached to the terminus of the PEG moiety. 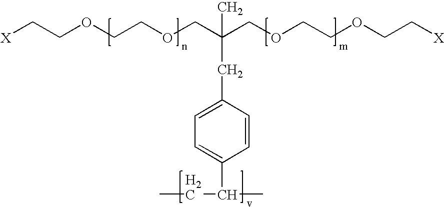
However, any suitable solid support useful for binding sample molecules and carrying out the desired chemistry and washing conditions can be used. The solid support can thus be glass, derivatized glass, silicon, plastic or other substrates. Any solid phase materials suitable for solid phase chemical synthesis are useful as solid supports in methods of the invention. The solid supports can be porous or non-porous materials, surface films, magnetic beads, colloids, membranes and the like. The solid supports can be in the form of beads, flat surfaces, or any configuration suitable for capturing molecules using the methods disclosed herein. The solid support can be derivatized to incorporate chemical moieties suitable for coupling to other chemical groups (e.g. linkers and tags, as discussed below) as desired.
Linkers

In certain embodiments, the present invention relates to use of linkers, for tethering a molecule to a solid support via a tag, which are stable to a wide range of conditions, but can be cleaved under well-defined conditions, thereby liberating said tagged molecule from the solid support. Preferred linkers will, when cleaved, form stabilized carbocations. Said stabilization can derive from substitution of the cationic center with aryl, preferably electron rich aryl, rings or alkyl groups, to form benzylic-like carbocations or tertiary carbocations.

In a preferred embodiment the linker is a NH₂-functionalized RINK linker, as shown below:

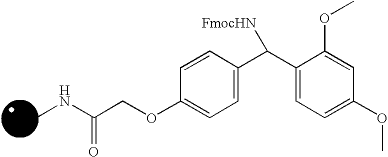

However, any suitable linker useful for connecting the tag to the solid support, which is also capable of being cleaved under acidic conditions, can be used. Examples of suitable liners are well know to ones skilled in the art and they include, for example, 4-hydroxymethyl-phenylacetamidomethyl linker (PAM linker; see Mitchell, A. R.; Erickson, B. W.; Ryabtsev, M. N.; Hidges, R. S.; Merrifield, R. J. Am. Chem. Soc. 1976, 98, 7357-7362.; Goldwasser, J. M.; Leznoff, C. C. Can. J. Chem. 1994, 56, 1562; and Zikox, C. C.; Ferderigos, N. G. Tetrahedron Lett. 1995, 36, 3741-3744): 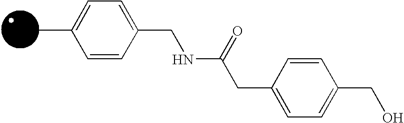
4-(1′,1′-Dimethyl-1′-hydroxypropyl)phenoxyacetyl linker (DHPP linker; see Akaji, K.; Kiso, Y.; Carpino, L. A. J. Chem. Soc. Chem. Commun. 1990, 584-586): 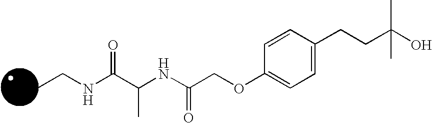
4-Hydroxymethylphenoxy linker (Wang linker; see Wang, S. J. Am. Chem. Soc. 1973, 95, 1328-1333; Floyd, C. D.; Lewis, C. N.; Patel, S. R.; Wittaker, M. Tetrahedron Lett. 1996, 37, 8045-8048; and Mathews, J.; Rivero, R. A. J Org. Chem. 1997, 62, 6090): 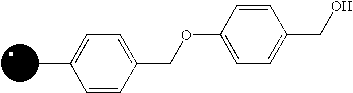
Super Acid Sensitive linker (SASRIN linker; see Mergler, M.; Tanner, R.; Gosteli, J.; Grogg, P. Tetrahedron Lett. 1988, 29, 32 4005-4008; and Nielsen, J.; Lyngso, L. O. Tetrahedron Lett. 1996, 37, 8439): 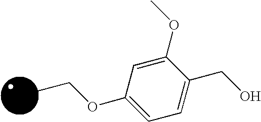
Hypersensitive Acid-Labile Tris(alkoxy)benzyl Ester linker (HAL linker; see Albericio, F.; Barany, G. Tetrahedron Lett. 1991, 32 (8), 1015-1018): 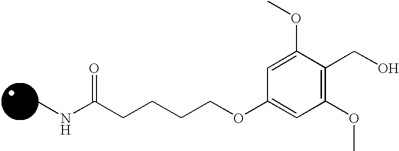
4-(2′,4′-dimethoxyphenyl-Fmoc-aminomethyl)-phenoxy linker (RINK linker; see Rink, H. Tetrahedron Lett. 1987, 28, 3787-3790; Beaver, K. A.; Siegmund, A. C.; Spear, K. L. Tetrahedron Lett. 1996, 37, 1145-1148; and Sutherlin, D. P.; Stark, T. M.; Hughes, R.; Armstrong, R. W. J. Org. Chem. 1996, 61, 8350): 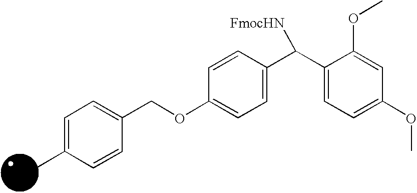
N-[9-(Hydroxymethyl)-2-fluorenyl]succinamic acid linker (HMFS linker; see Rabanal, F.; Giralt, E.; Albericio, F. Tetrahedron Lett. 1992, 33, 1775-1778; Rabanal, F.; Giralt, E.; Albericio, F. Tetrahedron Lett. 1995, 51, 1449-1458; and Eritja, R.; Robles, J.; Albericio, F.; Pederoso, E. Tetrahedron 1992, 40, 4171-4182): 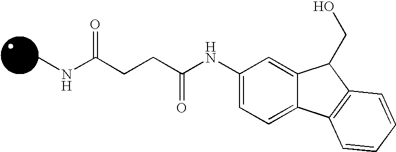
Fmoc-(Aminomethyl)-3,5-dimethoxyvaleric acid linker (Pal linker; see Albericio, F.; Kneib-Cordonier, N.; Biancalana, S.; Gera, L.; Masada, R. I.; Hudson, D.; Barany, G. J. Org. Chem. 1990, 55, 3730-3743; Landi, J. L.; Ramig, K. Synth. Commun. 1991, 21, 167-171; and Boojamra, C.; Burrow, K.; Ellman, J. A. J. Org. Chem. 1995, 60, 5742-5743. 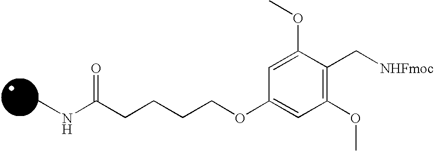
9-Xanthenyl linker (XAN linker; see Sieber, P. Tetrahedron Lett. 1987, 28, 2107-2110; and Chan, W. C.; Mellor, S. J. Chem. Soc., Chem. Commun. 1995, 1475-1477): 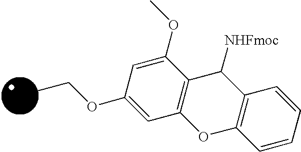
and the so-called Safety-Catch Acid-Labile linker (SCAL linker; see Patek, M.; Lebl, M. Tetrahedron Lett. 1991, 32, 3891-3894):

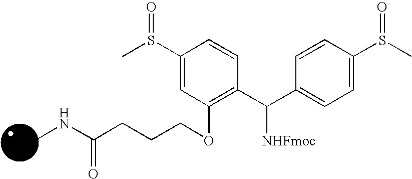

Other suitable linkers can be found in “The Combinatorial Index” by Barry A. Bunin, “Resins and Reagents” from Argonaut Technologies and “Combinatorial and Solid Phase Organic Chemistry” from Advanced ChemTech.

Tags

As used herein, a “tag” refers to a label that is detectable. The tag imparts a characteristic to a molecule such that it can be detected by any of a variety of analytical methods, including MS, chromatography, fluorography, spectrophotometry, immunological techniques, and the like. A tag can be, for example, an isotope, fluor, chromagen, ferromagnetic substance, luminescent tag, or an epitope tag recognized by an antibody or antibody fragment. A particularly useful tag is a mass tag, which is a mass label suitable for detection and analysis of a molecule by MS. Exemplary mass tags include, for example, a stable isotope tag, an isotope distribution tag, a charged amino acid, differentially isotopically labeled tags, and the like. A tag can also be a gas-phase basic group such as pyridyl or a hydrophobic group. A tag can also be an element having a characteristic isotope distribution, for example, chlorine, bromine, or any elements having distinguishable isotopic distribution. Additionally, a tag can have a bond that breaks in a collision cell or ion source of a mass spectrometer under appropriate conditions and produces a reporter ion.

For example, a particularly useful tag is a mass label useful for analysis of a sample by MS. The change in mass of the molecule due to the incorporation of a mass label should be within the sensitivity range of the instrument selected for mass determination. In addition, one skilled in the art will know or can determine the appropriate mass of a label for molecules of different sizes and different compositions. Moreover, when using heavy and light mass labels, for example, for differential labeling of molecules, a mass difference as small as between about 1-3 mass units can be used or as large as greater than about 10 mass units, for example, about 4, about 5, about 6, about 7, about 8, about 9, about 10, about 12, about 15, or about 20 mass units or greater, as desired.

In a preferred embodiment, the tags incorporate deuterium. Examples of such tags are shown in FIG. 1B. In all embodiments, the tags include a nucleophilic moiety, as described below.

Nucleophiles

The term “nucleophile” is recognized in the art, and as used herein means a chemical moiety having a reactive pair of electrons. Examples of nucleophiles include uncharged compounds such as water, amines, mercaptans and alcohols, and charged moieties such as alkoxides, thiolates, carbanions, and a variety of organic and inorganic anions. Illustrative anionic nucleophiles include simple anions such as hydroxide, azide, cyanide, thiocyanate, acetate, formate or chloroformate, and bisulfite. Organometallic reagents such as organocuprates, organozincs, organolithiums, Grignard reagents, enolates, acetylides, and the like may, under appropriate reaction conditions, be suitable nucleophiles.

The term “electrophile” is art-recognized and refers to chemical moieties which can accept a pair of electrons from a nucleophile as defined above. Electrophiles useful in the method of the present invention include cyclic compounds such as epoxides, aziridines, episulfides, cyclic sulfates, carbonates, lactones, lactams and the like. Non-cyclic electrophiles include α,β-unsaturated carbonyls, sulfates, sulfonates (e.g. tosylates), chlorides, bromides, iodides, and the like.

Nucleophiles which are useful in the present invention may be determined by the skilled artisan according to several criteria. In general, a suitable nucleophile will have one or more of the following properties: (1) It will be capable of reaction with the substrate at the desired electrophilic site; (2) It will yield a useful product upon reaction with the substrate; (3) It will not react with the substrate at functionalities other than the desired electrophilic site; and (4) It will not substantially undergo further undesired reaction after reacting with the substrate in the desired sense. It will be understood that while undesirable side reactions may occur, the rates of such reactions can be rendered slow—through the selection of reactants and conditions—in comparison with the rate of the desired reaction(s).

For example, if a nitrogen-containing nucleophile is desired, it may be selected from ammonia, phthalimide, hydrazine, an amine or the like. Similarly, oxygen nucleophiles such as water, hydroxide, alcohols, alkoxides, siloxanes, carboxylates, or peroxides may be used to introduce oxygen; and, in a preferred embodiment, mercaptans, thiolates, bisulfite, thiocyanate and the like may be used to introduce a sulfur-containing moiety. Additional nucleophiles will be apparent to those of ordinary skill in the art.

For nucleophiles which exist as anions, the counterion can be any of a variety of conventional cations, including alkali and alkaline earth metal cations and ammonium cations.

Application of the Invention to a Particular Biochemical System

On embodiment of the solid-phase Michael addition (SMA) method for isolation of phospho-peptides is illustrated in the Figures. Briefly, cysteine residues of tryptic phospho-peptides were reduced and alkylated. The key chemical reactions, β-elimination and solid-phase Michael addition, occur under alkaline reaction conditions. Phospho-serine and -threonine were converted to dehydroalanine and dehydrobutyric acid, respectively. Newly formed double bonds between the α and β carbons of dehydroalanine/dehydrobutyric acid subsequently served as Michael acceptors with which nucleophilic resins react. In contrast to previously reported liquid-phase Michael addition-based methods, phospho-peptides were directly captured by the solid Michael-addition resins. Unreacted peptides and debris were then washed away while phospho-peptides formed a covalent linkage and remained on the resin. Captured peptides in tagged form were released by trifluoroacetic acid-mediated cleavage of the amine bond. The chemical structure of the nucleophilic group used in the resin is depicted in the Figures. The negatively charged phosphate is substituted with an amide-containing tag. The molecular mass of the tag moiety is 203.09 Daltons and the delta mass at serine/threonine residues is 186.12 Daltons. Phosphorylation sites on released products were subsequently determined by liquid chromatography-coupled tandem mass spectrometry.

A phospho-Ser peptide derived from the cyclic-AMP responding element binding (CREB) protein was captured using the methods of the invention. After SMA treatment, the chemically tagged products were analyzed by matrix-assisted laser desorption/absorption ionization-time of flight mass spectrometry (MALDI-TOF MS). We found that the major molecular ions at mass/charge (m/z) 1796.97, representing the phosphorylated form of the CREB peptide, shifted to a higher m/z value of 1903.02. This value matched the theoretical m/z value of the tagged-CREB peptide, suggesting that the phosphate-containing CREB peptide was captured by the resin and the phosphate group of the peptide was replaced with the tag moiety after the cleavage step. A side product at m/z 2103.05 was also detected.

Similar CREB peptide capture experiments have been performed on four different resin preparations. To investigate the specificity of SMA, we attempted to isolate the phospho-CREB peptide (1 pmol) from a non-phosphoryl peptide library with KSYGXEXTLXAE sequences (X=any amino acids except cysteine, 6859 different peptides in total, approximate weight ratio of the peptide library to the CREB peptide=8000:1). We found that SMA was able to specifically capture and tag the phospho-CREB peptide as judged by MALDI-TOF analysis. Remarkably, SMA isolated none of non-phosphoryl peptides. In addition to phospho-Ser containing peptides, we found that our method was able to capture and tag a synthetic phospho-Thr peptide with a VDAAV(pT)PEERH(carbamidomethyl-C) sequence.

In addition to assessing the specificity of SMA, the yield of capturing phospho-peptides was also evaluated. One nmol of CREB phospho-peptide was incubated with 50 μL of resin (provides approximately 10 μmol of functionalized ligand). Upon treatment with SMA, all of the input peptide (1 nmol) was efficiently recovered. No phospho- and dehydro-form of the CREB peptides were detected in the unbound fraction. To test the sensitivity of SMA, 50 μL of the SMA resin was incubated with various amounts of phospho-CREB peptide. The resin was able to recovery synthetic phospho-peptides from concentrations as low as 250 fmol of pure CREB phospho-peptide as judged in MALDI-TOF measurement. Together, these results suggest that the thio-nucleophilic resin can specifically capture, release, and tag phospho-serine/threonine containing peptides.

To demonstrate the SMA chemistry and its application for acquiring biological information from complex samples, we focused on the CDK5-mediated phosphorylation of microtubule-associated proteins. The longest isoform of the human microtubule-associated protein Tau is composed of 441 amino acid residues, among which 45 residues are serine and 35 are threonine. Phosphorylation events regulate the interaction between Tau and microtubule and facilitate the formation of neurofibrillary tangles in Alzheimer's disease (AD). At least 29 sites on AD-Tau are phosphorylated by a variety of serine/threonine kinases including PKA, ERKs, GSK3β, and CDK5. Currently, research on Tau phosphorylation is restricted to a limited number of phosphorylation-specific antibodies which cannot reveal the heterogeneous phosphorylation status of Tau. Therefore, analysis of global Tau phosphorylation states by improved proteomic methods is desirable as it will contribute to a better understanding of the mechanism underlying AD and offer potential insight into therapeutic intervention of the disease.

CDK5 is a proline-directed protein kinase essential for development of the mammalian central nervous system. CDK5 requires association with a regulatory activator p35, a neuronal specific protein, to become active. Calpain-dependent cleavage of p35 to p25 has been implicated in the pathogenesis of AD. While CDK5/p35 is a poor Tau kinase, CDK5/p25 has been shown to cause hyperphosphorylation of Tau. To determine the phosphorylation sites of CDK5-phosphorylated Tau using SMA, recombinant Tau protein was phosphorylated in vitro by purified CDK5/p25 to a stoichiometry of 0.5 phosphates per Tau molecule. Tryptic peptides derived from the unphosphorylated or phosphorylated Tau protein were first analyzed by MALDI-TOF MS. We found one peak at m/z=1473.60 which matches the mass of a monophospho-peptide SGYSSPGSPGTPGSR in samples phosphorylated by CDK5. This monophospho-peptide did not appear in the control sample without kinase incubation. The signal intensity of the phospho-peptide diminished after phosphatase treatment. The tryptic peptide samples containing phosphorylated and dephosphorylated peptides were further analyzed by SMA. One molecular ion corresponding to the mass of SGYSSPGSPGTPGSR plus one tag (m/z 1579.72) was observed in the phosphorylated sample but not in the dephosphorylated sample. A previous report showed that minor β-elimination may occur at serine/threonine residues in the absence of phosphates, however, we did not observe such side reactions on the dephosphorylated SGYSSPGSPGTPGSR peptides. These data suggest that SMA isolated the phosphorylated form of the peptide, but not the unphosphorylated form. In addition to isolating peptides, SMA was also tested for isolation of phospho-Tau proteins.

IMAC has being applied to enrich for phospho-peptides. To compare IMAC and SMA, we used an iron (III)-nitriloacetic acid gel (an IMAC) to enrich for phosphorylated peptides from the CDK5-phosphorylated Tau tryptic peptide pool shown in the Figures. A MALDI-TOF spectrum of IMAC-isolated peptides showed that IMAC was able to isolate the same SGYSSPGSPGTPGSR phospho-peptide isolated by SMA. These results suggest that both SMA and IMAC can enrich for phosphorylated peptides in a complex system. However, three acidic residue-rich non-phospho-peptides QEFEVMEDHAGTYGLGDR (m/z 2053.89), DQGGYTMHQDQEGDTDAGLK (m/z 2165.90), and KDQGGYTMHQDQEGDTDAGLK (m/z 2293.99) were also enriched by the IMAC approach. This may be due to the preferential enrichment of acidic peptides by IMAC. The overall complexity of recovered peptides by SMA is less than that obtained by IMAC. Thus, one significant advantage of SMA over IMAC is that it yields less background unphosphorylated peptides after enrichment, increasing the probability of detecting low-abundance phosphorylation events.

We further used tandem mass spectrometry to analyze the SMA-treated Tau peptides in order to gain sequence information from fragmented peptide ions. A collision-induced-dissociation spectrum obtained from an ion-trap instrument showed a parent ion of m/z 790.35 (2+) containing the SGYSSPG (tag-S)PGTPGSR peptide. The same molecular ion was also detected and analyzed in a quadrupole-time of flight instrument. A quadrupole-time of flight (TOF) ms/ms spectrum was acquired and the same peptide sequence was detected as that detected from the ion-trap MS spectrum. Most of the b series ions and y series ions of the peptide are detected in both instruments. Three major daughter ions y4, y7, and y10 were detected in the spectrum, indicating the presence of proline-induced fragmentation. This suggests that the chemical tag does not influence peptide fragmentation.

To further verify the sites on Tau phosphorylated by CDK5, we used presently available phospho-Tau antibodies to probe in vitro phosphorylated Tau. AT-8, which is raised against phospho-Ser202/Thr205 of Tau, recognized in vitro CDK5-phosphorylated Tau. Conversely, Thr231 of Tau is not a ideal substrate for CDK5, and neither SMA nor the phosphorylation specific antibody pT231 was able to detect the phosphorylation of this residue by CDK5. Thus, these phospho-epitope specific antibodies confirm the SMA-determined phosphorylation status of Tau.

In order to study CDK5-induced Tau phosphorylation in an even more biologically relevant context, we transfected human Tau with or without the active CDK5 complex in neuroblastoma CAD cells. Heat-stable Tau samples prepared from transfected cells were treated with SMA followed by tandem mass spectrometric analysis. We found that residues Thr181, Ser185, Ser191, Ser195, Ser198, Ser199, Ser202, Thr205, Ser208, Ser210, Thr217, Thr220, Ser241, Ser396, and Thr403 were phosphorylated (Table1). Among these residues, Thr181, Ser199, Ser202, Thr205, Thr217, and Ser396 are (S/T)P sites representing the consensus CDK5-phosphorylation motif and are previously reported CDK5 sites. Immunoblots using phospho-Tau antibodies show that phosphorylation of Ser202, Thr205, and Ser396 was significantly increased, whereas no phosphorylation of Thr231 was detected in the sample prepared from CDK5/p25/Tau triple transfected CAD cells, supporting results obtained by SMA followed by mass spectrometry (Table 1). Most phospho-Tau peptides listed in Table 1 were not detected in the PKA-Tau transfected controls (phosphorylation control) or in an un-functionalized resin control (non-specific binding control).

The detection of some phospho-peptides that do not correspond to the CDK5 consensus phosphorylation motif suggests that over-expressed Tau in CAD cells is also phosphorylated by other Ser/Thr kinases (Table 1). A previous study also shows that CDK5 activation can lead to activation of non-proline-directed kinases. Some peptides were multiply tagged, suggesting that the resin can label multiple phosphorylation sites on the same peptide. The presence of disulfide-linked tag dimers after the acid cleavage suggests that the spatial proximity of adjacent ligands allows the reactions of multiple tags on one peptide to occur. We note that not all phosphates were replaced by the chemical tag. This suggests that β-elimination is influenced by the sequence, composition, and structure of peptides. Nevertheless, both the tagged and phosphorylated forms of serine and threonine can be simultaneously identified by tandem mass spectrometry.

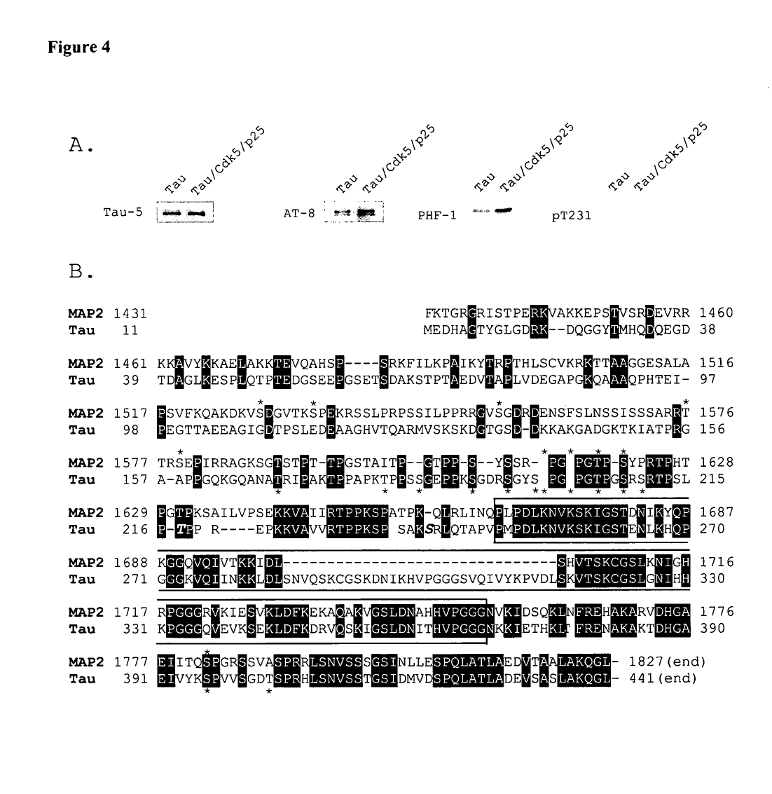

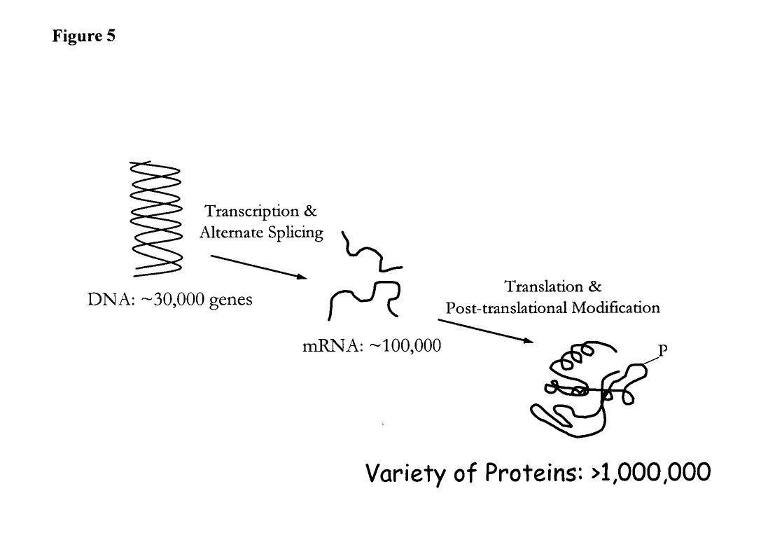

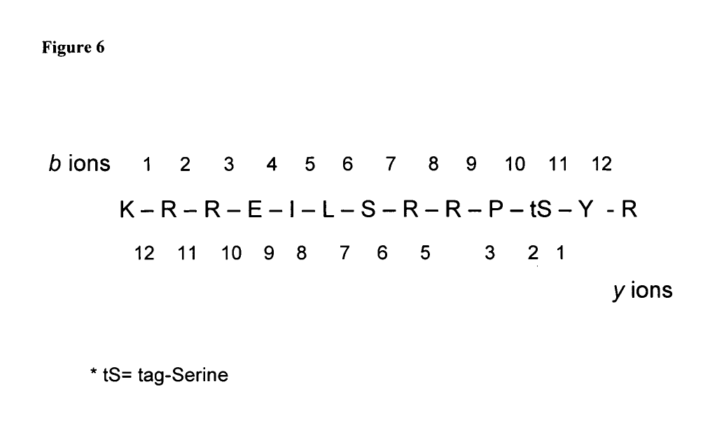

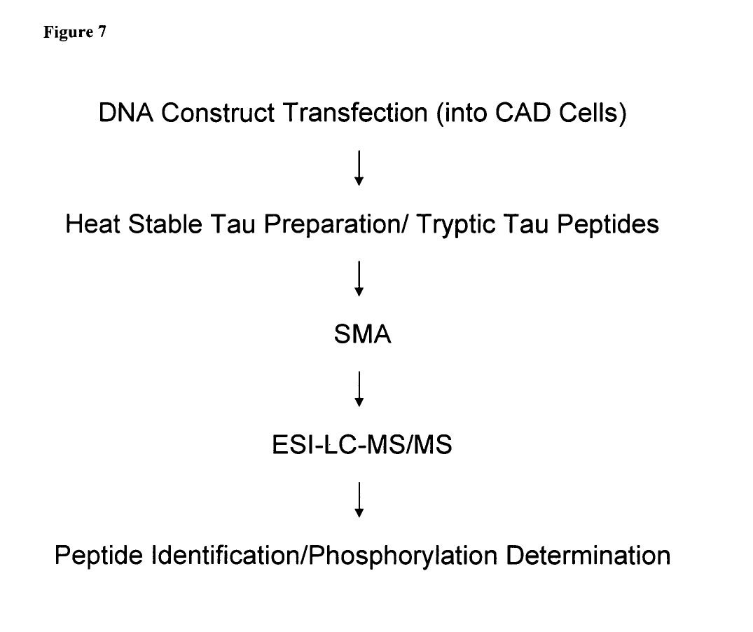

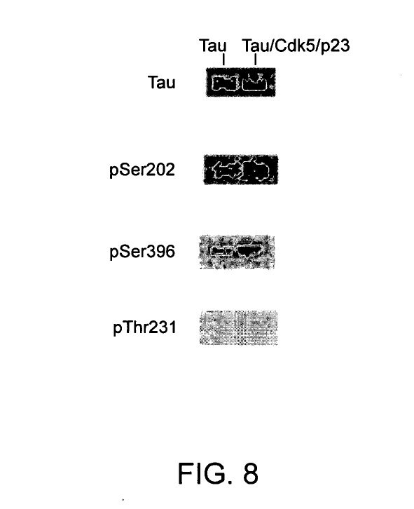

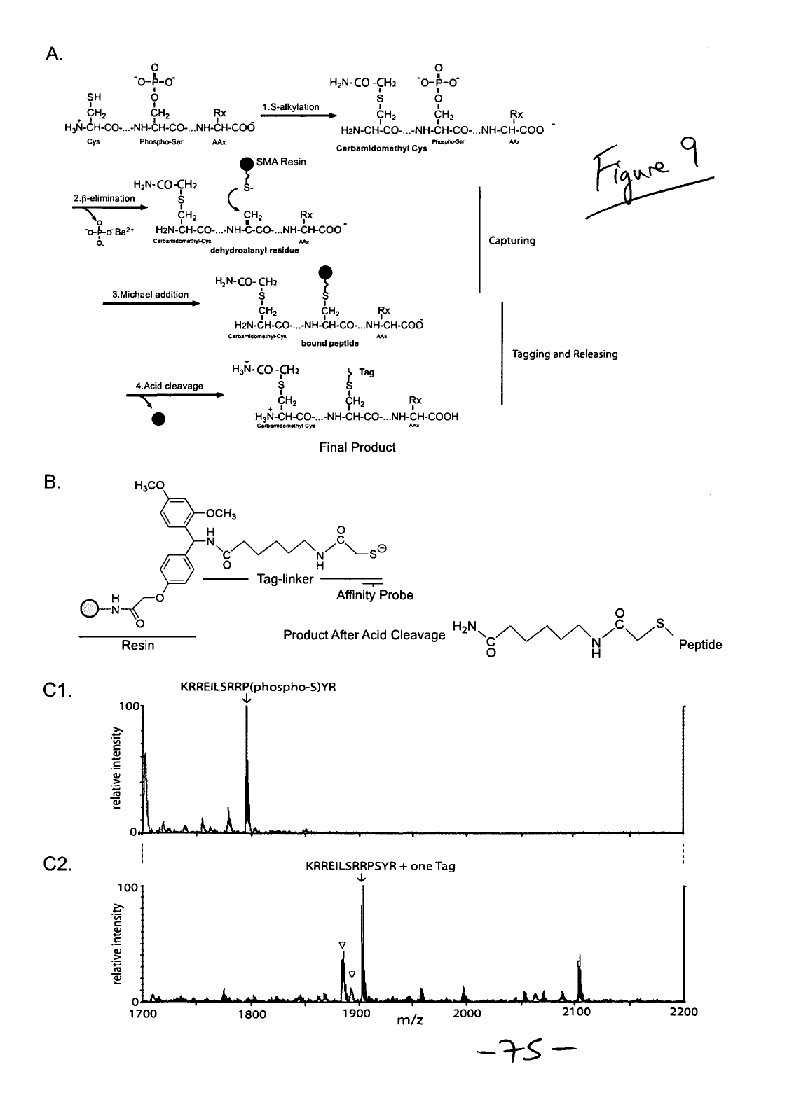

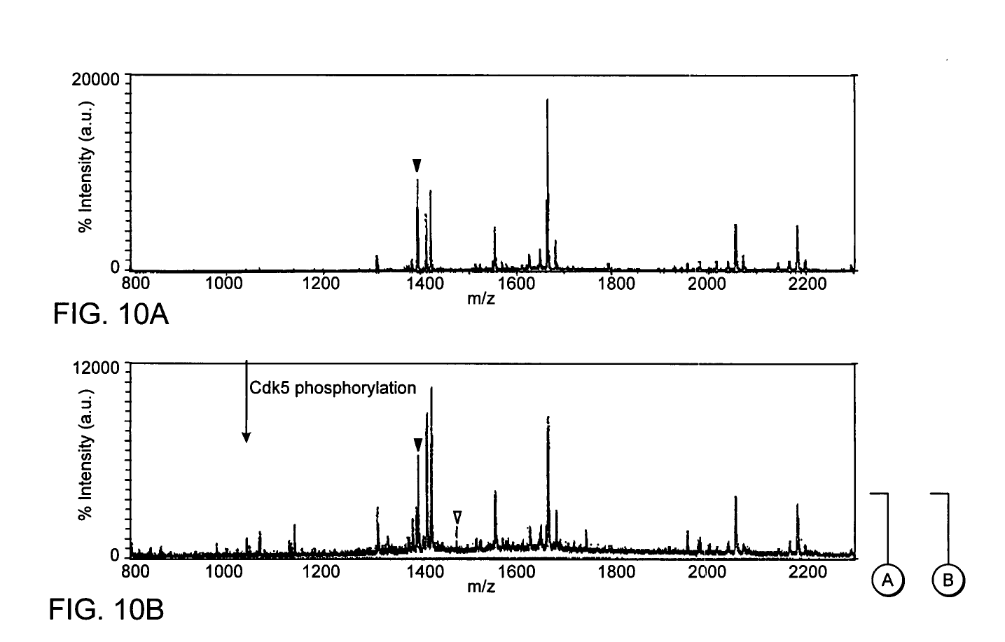

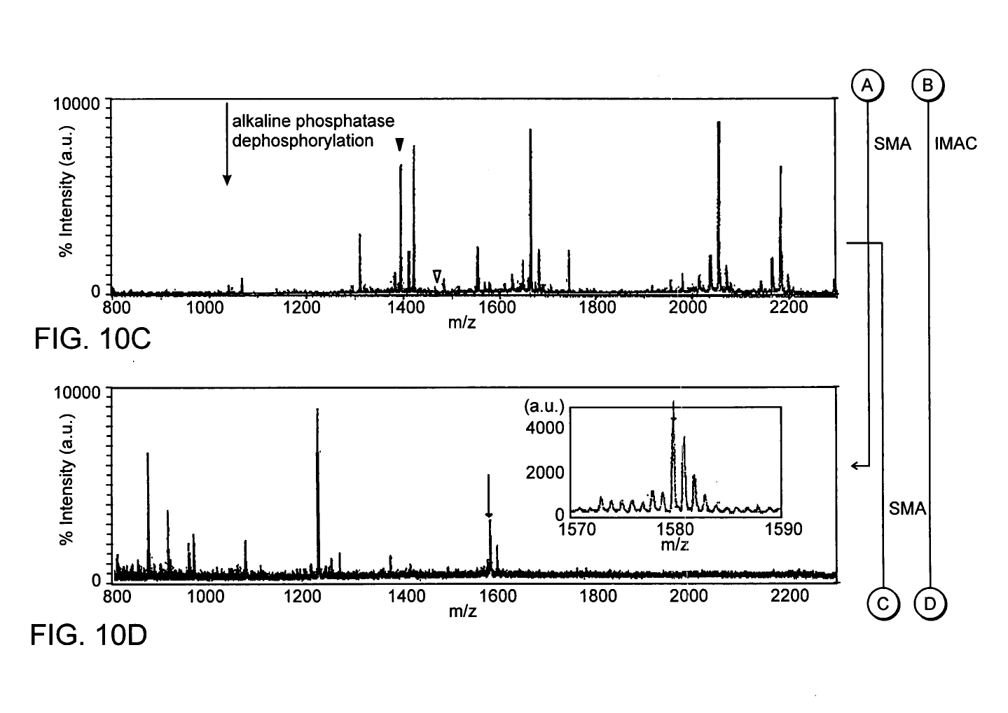

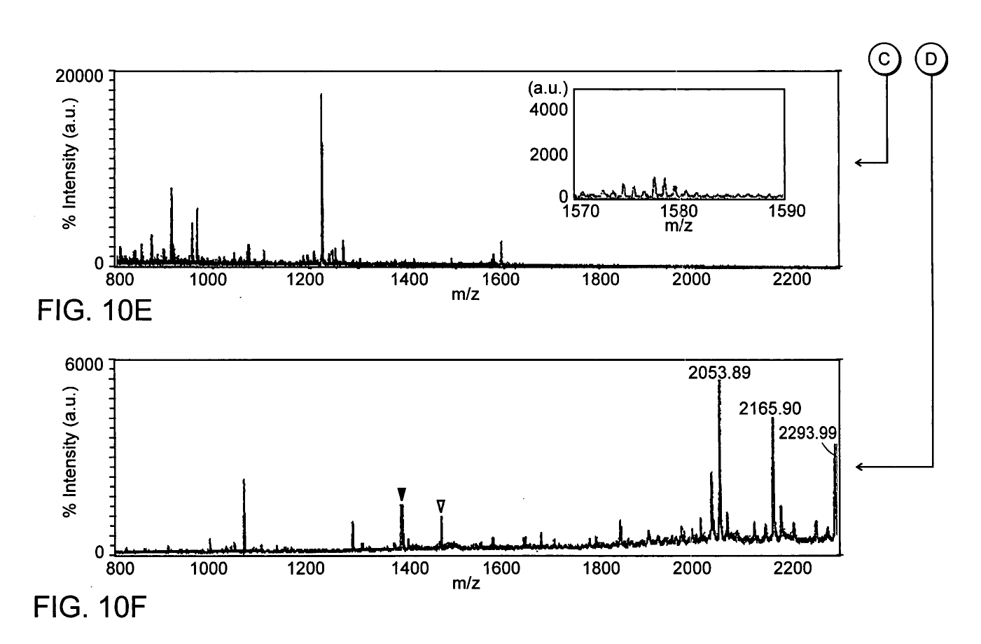

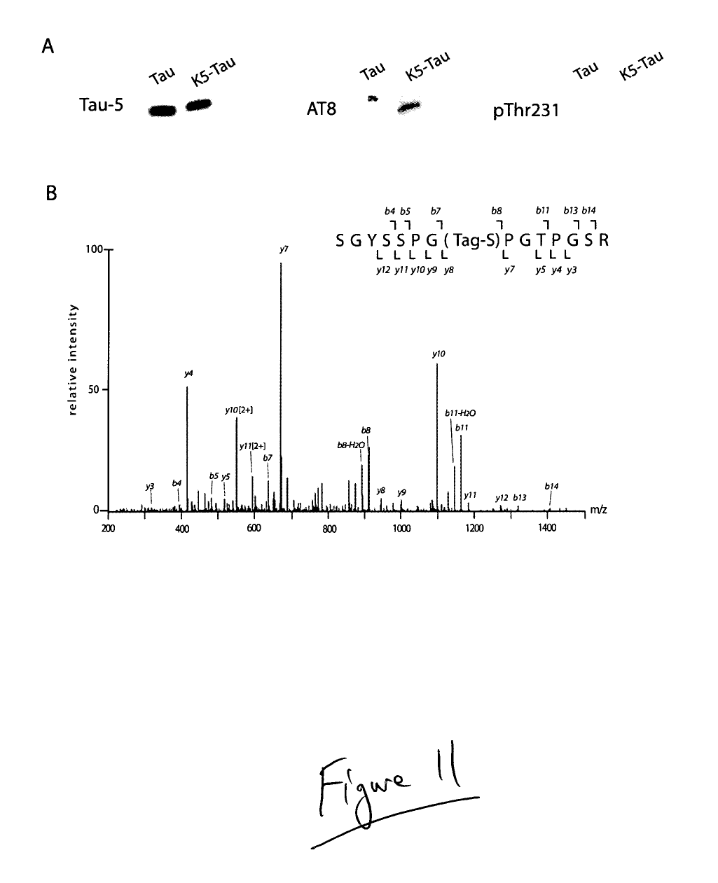

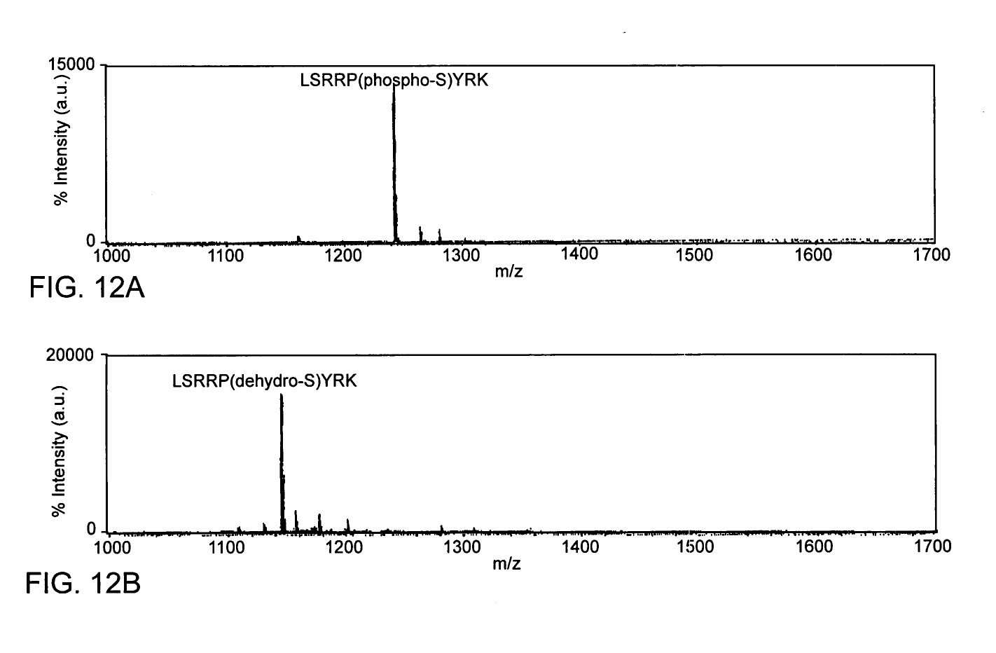

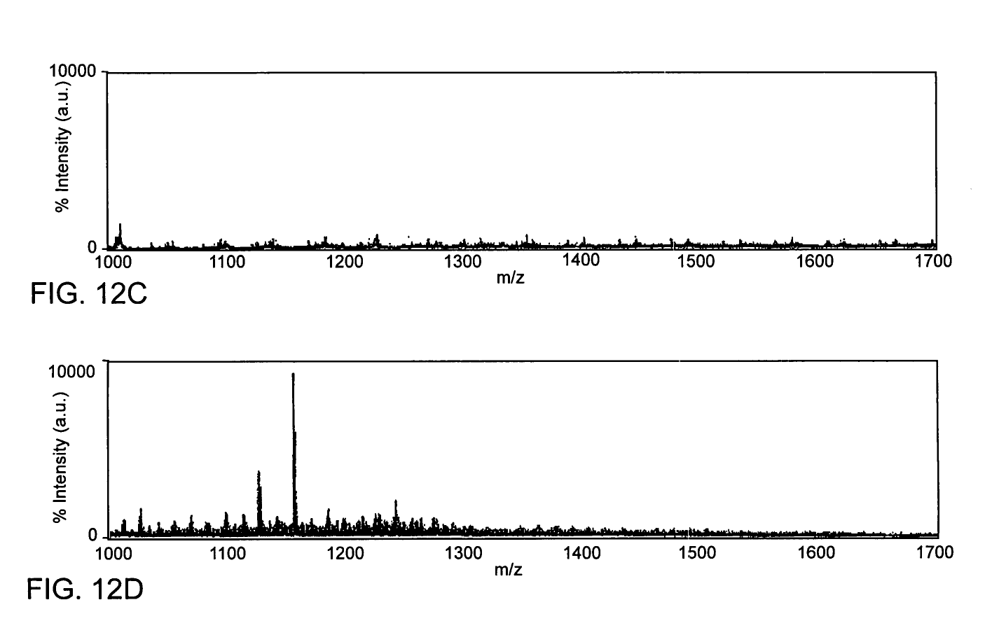

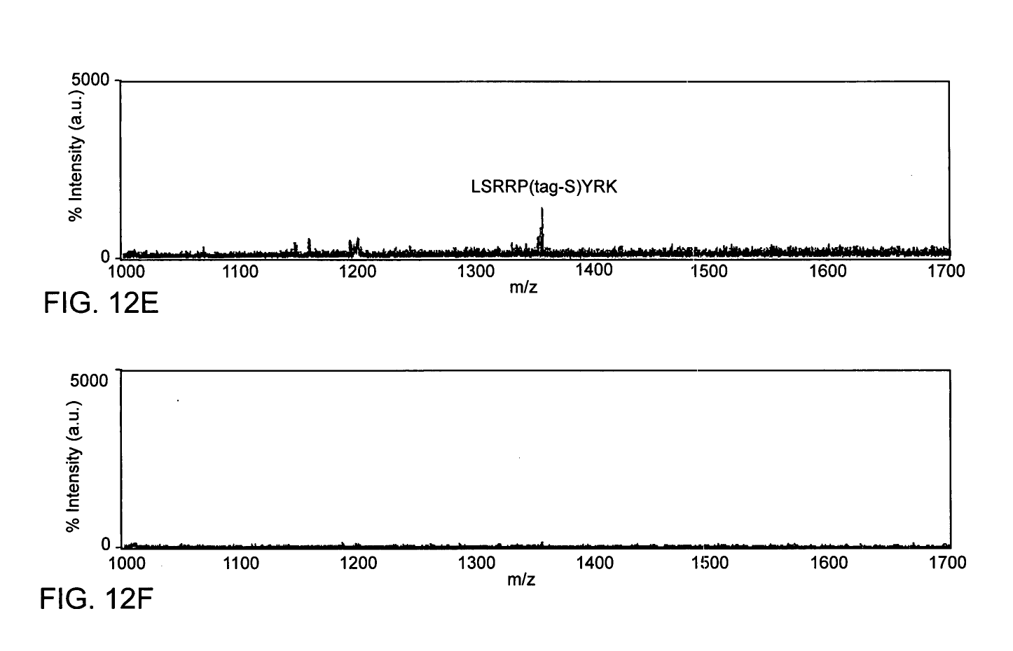

In addition to phospho-Tau peptides, we also detected phospho-peptides derived from other cellular proteins in CAD cells. Among these proteins, we discovered a phosphopeptide corresponding to microtubule associated protein 2 (MAP2), a potentially novel CDK5 substrate. We decided to focus on MAP2 because it shares nearly identical microtubule-binding repeats and a proline-rich region with Tau. Like Tau, MAP2 is also phosphorylated by multiple kinases. However, information on the specific phosphorylation sites are limited and it is not know whether CDK5 phosphorylates MAP2 in cells. Using SMA, we found that endogenous MAP2 was phosphorylated in cells transfected with Tau alone (Table 1). This supports a previous study that MAP2 is constitutively phosphorylated in vivo. In the control sample, phosphorylation at residues Thr1452, Thr1507, Ser1514, Ser1519, Ser1528, and Ser1555 was detected (Table 1). In CDK5-active cells, residues Ser1528, Ser1534, Ser1555, Thr1613, Thr1616, Thr1619, Ser1621, and Ser1782 were phosphorylated (Table 1). Interestingly, in CDK5/p25-expressing cells, specific phosphorylation of MAP2 was limited to (S/T)P sites (Ser1534, Thr1613, Thr1616, Thr1619, and Ser1782 in Table 1) whereas phosphorylation events at Ser1528 and Ser1555 appeared in both control and CDK5-activated cells. Among these (S/T)P phosphorylation sites, Thr1613/Thr1616/Thr1619, and Ser1782 on MAP2 are at analogous regions of Ser199/Ser202/Thr205 and Ser396 on Tau, the two main CDK5 phosphorylation modules of Tau (FIG. 4B). The sequence 1613TPGTPGTP1620 is located in the proline-rich region of MAP2, and phosphorylation of this proline-rich region was observed in growth regions of rat hippocampal neurons. In an in vitro phosphorylation experiment, purified CDK5/p25 was able to phosphorylate (S/T)PG(S/T)PGTP peptides and purified MAP2 (data not shown). This suggests that SMA was able to identify a novel CDK5 substrate which was further confirmed by in vitro phosphorylation.

Previous studies showed that CDK5 preferably phosphorylates (S/T)PX(K/H/R) sequences. Our results suggest that 1613TPGTPGTP1620 of MAP2 and 199SPGSPGTP206 of Tau may represent another favorable CDK5 phosphorylation motif, (S/T)PG(S/T)PGTP. Furthermore, this motif features two consecutive PXXP sequences which represent SH3 domain binding sites. Phosphorylation of this motif influences the binding of the SH3 domain in protein tyrosine kinase Fyn to Tau and the binding of prolyl isomerase Pin1 to Tau. The binding of SH3 domain containing Src to MAP2 was inhibited by phosphorylation of proline-directed kinase mitogen-activated protein kinase and extracellular signal-regulated kinase 2 not by that of PKA.

To understand the basal phosphorylation status of the heat-stable proteins in vivo, including microtubule-associated proteins, we treated 250 μg of tryptic peptides derived from the heat-stable fraction of mouse forebrains with SMA. We detected 114 phospho-peptides with 184 phosphorylation sites (Supplementary Table) from 999 MS/MS spectra. The majority of recovered phospho-peptides were mono-phospho-peptides (41%) and di-phospho-peptides (46%). Tri-phospho-peptides and quadru-phospho-peptides encompass 12% in total. One Tau peptide, 526SPSASKSR533 (numbering based 732 amino acid-long Tau) was tagged at residues Ser235 and Ser241 (numbering based on 441 amino acid-long Tau). The phosphorylation sites on the MAP2 peptide, 1650TPPKSPATPK1659, corresponds to the phosphorylation of Thr1649, Ser1653, and Thr1656 in human MAP2.

Overall, we have presented evidence that our solid-phase procedure is able efficiently to isolate and identify phospho-peptides from whole cell systems. There are several notable advantages over previously published procedures. First, the labeling of phosphorylation sites and enrichment of phospho-peptides by our method occurs in a single solid-phase one pot reaction which may reduce the likelihood of sample loss. This one pot reaction is different from previous attempts which perform β-elimination and Michael addition in the liquid phase, enrich products with solid-phase resins, and subsequently recover peptides. Second, the mass of the 6-(2-mercapto-acetylamine)-hexanoic amide tag, which is smaller than a previously described biotin tag, allows for a wider detection window in the mass spectrometric measurement. Third, a previous study reports that replacement of a phosphate with an amine-containing moiety at phosphorylation sites can enhance phospho-peptide detection in mass spectrometry. We detected no neutral-loss of the 6-(2-mercapto-acetylamine)-hexanoic amide tag by MALDI-TOF MS analysis (FIGS. 1C2 and 2D) or by tandem mass spectrometric analysis, suggesting that the tag is a more stable moiety than a phosphate. Fourth, we use barium hydroxide, which is more efficient than sodium hydroxide or potassium hydroxide reported previously, to catalyze β-elimination. Beta-elimination may also occur on carbamidomethylated cysteinyl or O-linked glycosylated peptides. In our hands, carbamidomethyl cysteinyl residues remain intact and do not undergo β-elimination. The use of barium hydroxide can significantly reduce the reaction rate of β-elimination of O-linked glycosylated peptides by two orders of magnitude compared to that of phospho-Ser/Thr peptides. Thus these potential side reactions should be very minor during SMA.

Dephosphorylation of phospho-peptides with phosphatases prior to SMA may serve as an additional control to assure accurate phosphorylation determination. Recently, Collins et al. showed that different IMAC protocols yield different coverage in phosphorylation site determination. One of our preliminary phospho-proteomic studies on mouse brains suggests that phospho-peptides recovered by SMA and IMAC are not entirely identical. It is likely that different methods of capturing phospho-peptides enrich different groups of phospho-peptides. A study of phosphorylation events on synaptic proteins showed that 351 phospho-peptides were recovered from approximately 18 mg of sample by IMAC approaches. Classic IMAC and improved IMAC likely isolate peptides with multiple phosphorylation sites. An application of strong cation exchange chromatography combined with neutral-loss detection of phosphoric acid can detect more than two thousand phosphorylation sites among 525,000 MS/MS spectra when 8 mg of starting materials was used. This strong cation exchange method prefers the isolation of monophospho-peptides. In contrast, SMA is designed to target β-elimination-sensitive phosphorylation sites. The covalent bond linkage of the phospho-peptide to SMA resins is more stable during the non-phospho-peptide removal step than the ionic linkage in IMAC or strong cation exchange. SMA is expected to recover a wide range of phospho-peptides and yield a higher percentage of phospho-peptides. The phospho-proteomic analysis of the mouse heat-stable proteins supports this expectation.

Mass Spectrometry Studies

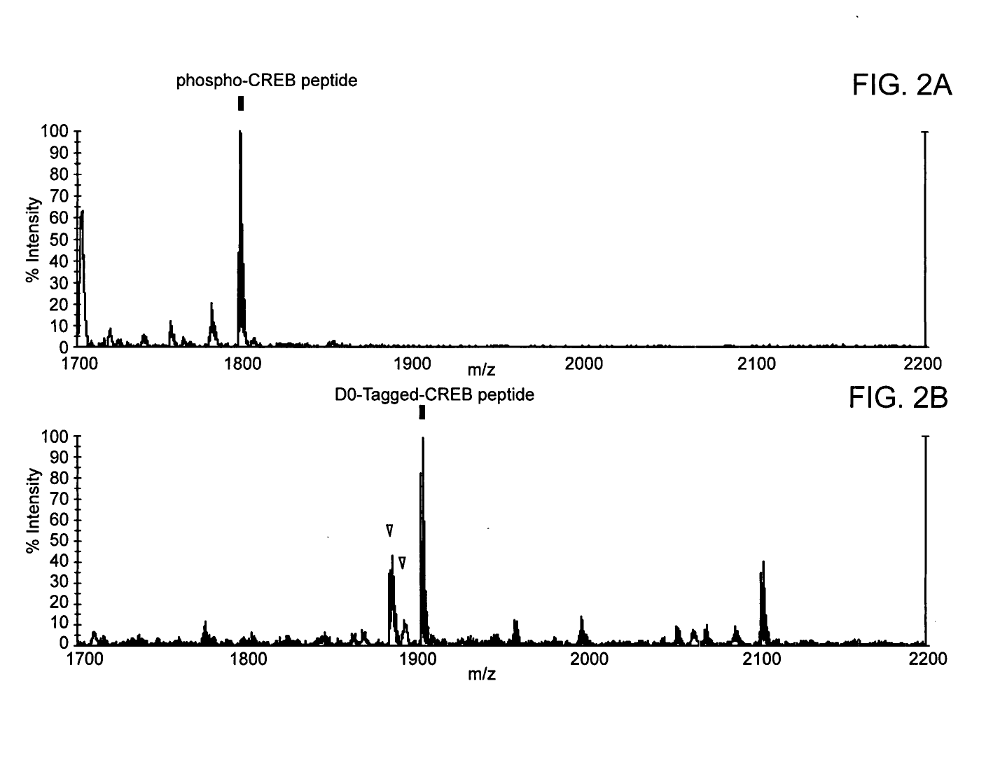

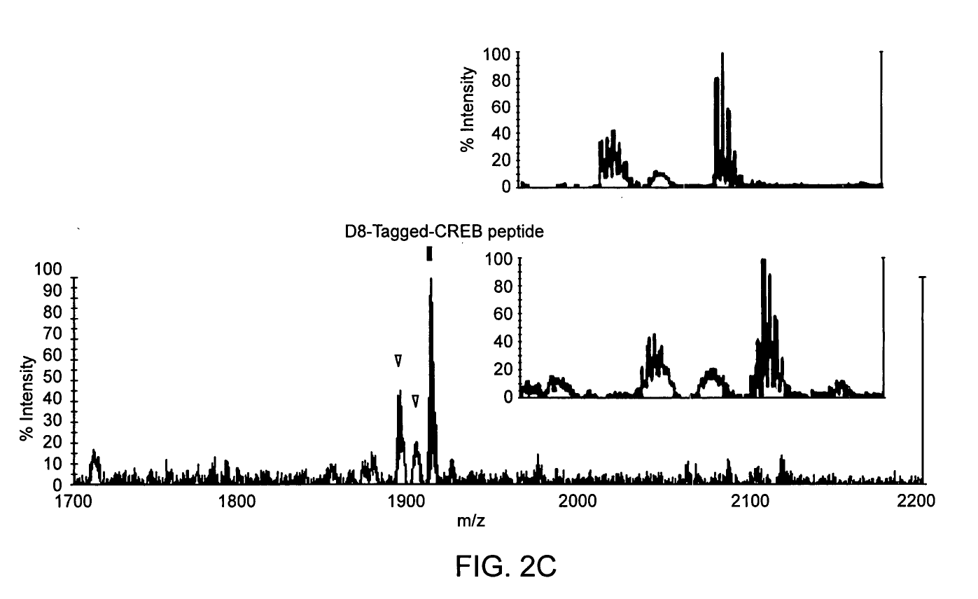

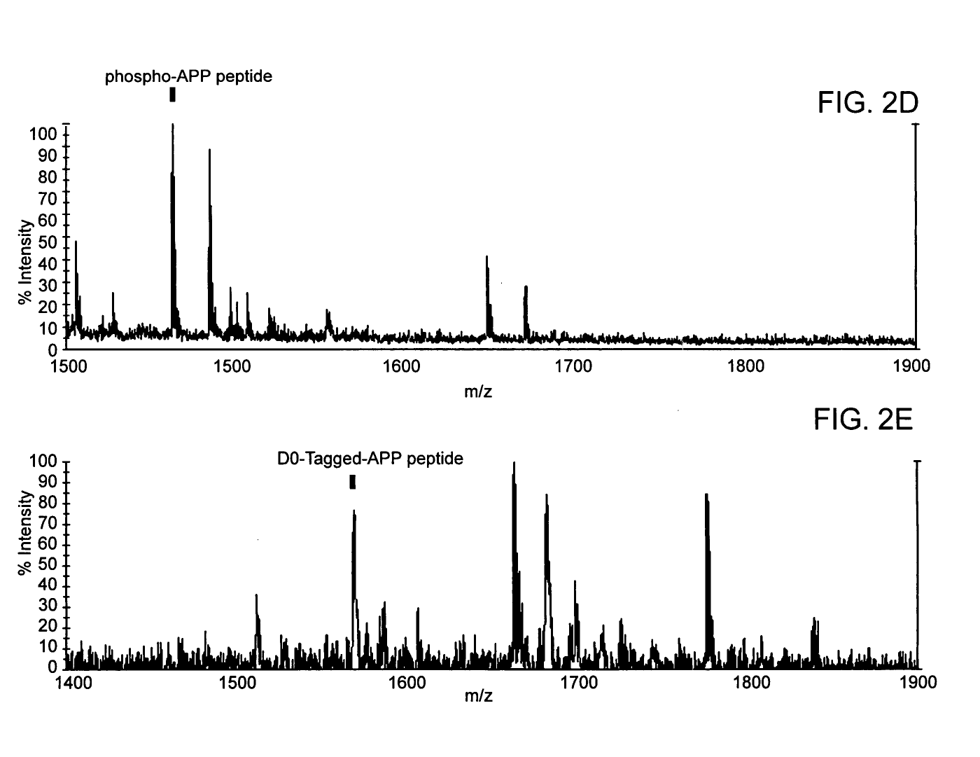

To determine whether the resin can capture phospho-peptides, a phospho-Ser CREB (cyclic-AMP responding element binding) peptide with an amino acid sequence of H₂N—KRREILSRRP(phospho-S)YR—OH was tested. After the chemical treatment, cleaved products were analyzed by matrix-assisted laser desorption/absorption ionization-time of flight (MALDI-TOF) mass spectrometry. The major molecular ions (mass/charge, m/z 1798) representing the phosphorylated form of the CREB peptide (FIG. 2A) shifted to a higher m/z position (m/z 1902) after release from the resin (FIG. 2B). The m/z value of 1902 matched the theoretical m/z value of tagged-CREB peptides (FIG. 2B). This result suggested phospho-CREB was captured by the resin and the phosphate group on the peptide was replaced with the tag moiety after the acid cleavage step. To further provide evidence that tag was present on the released peptide, a deuterium-loaded 6-(2-mercapto-acetylamide)-hexanoic amide resin (FIG. 1B) was generated to probe phospho-CREB peptides. The captured peptides appeared 8 Daltons heavier (m/z 1910) than the undeuterated-tagged peptides (m/z 1902) (FIG. 2C). This data indicates that both molecular ions, m/z 1902 in FIG. 2B and m/z 1910 in FIG. 2C, possess the tag. Analysis of the isotopic peak distribution revealed the presence of molecular ions containing 7 deuterium atoms due to proton-deuterium exchange at the a position. In addition to capturing and releasing purified synthetic peptide, the method was able to isolate the phospho-CREB peptide from a non-phosphoryl peptide library with KSYGXEXTLXAE sequences (X=any amino acids except cysteine, 6859 different peptides in total) as judged by a MALDI-TOF analysis (data not shown). Minor background binding of non-phosphoryl peptides to the resin was also detected.

To determine whether this method is applicable for isolating phospho-Thr peptides, a synthetic phospho-Thr peptide (VDAAV(phospho-T)PEERHC) derived from the amyloid precursor protein (APP) was tested. MALDI-TOF spectra of the phospho-Thr peptide before and after the chemical treatment are shown in FIGS. 2D and E, respectively. A peak at m/z 1568 matched the theoretic value of the tagged-Thr peptide. This data demonstrates that the resin is capable of capturing phospho-Thr peptides.

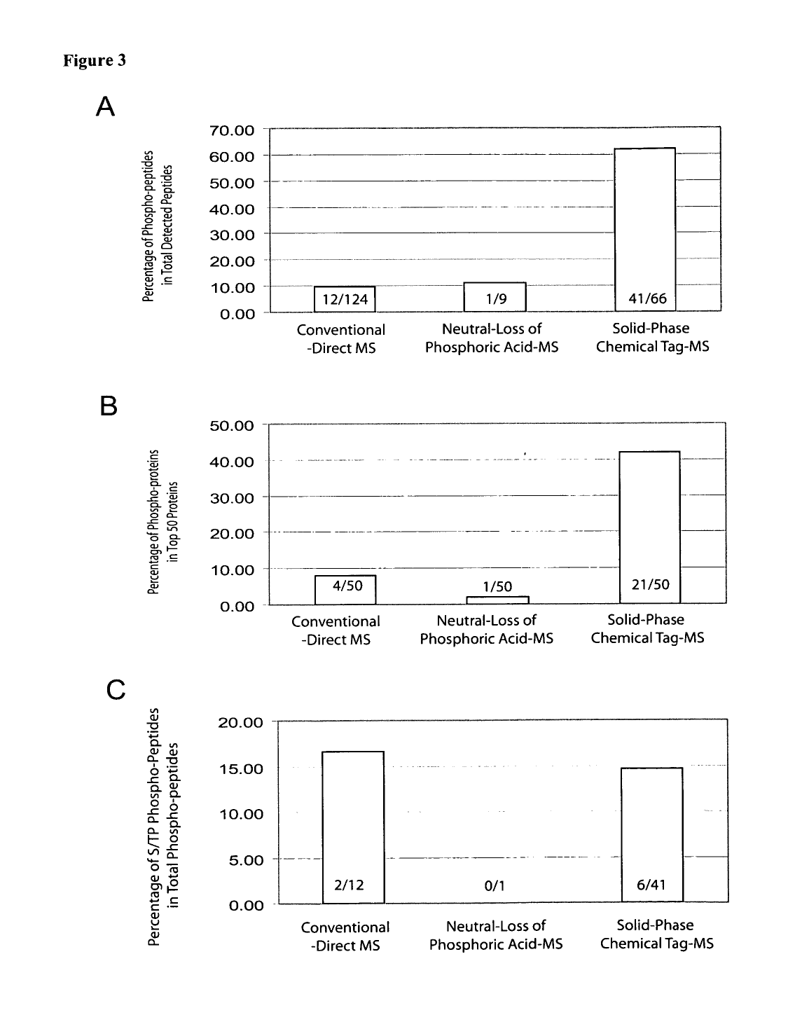

To determine the efficiency of enriching phospho-peptides using the solid-phase chemical tagging approach, phospho-peptide data obtained from three different tandem mass spectrometric protocols: conventional MS/MS, neutral-loss of phosphoric acid MS/MS, and the solid-phase chemical tagging MS/MS were compared. Tryptic peptides prepared from mouse brain cortical synaptosomal membrane proteins were used in this method assessment. In conventional MS and neutral-loss MS measurements, approximately 10% of molecular ions were phospho-peptides (FIG. 3A). In contrast, efficiency of phospho-peptide recovery was significantly increased to 60% with the solid-phase phospho-peptide enrichment (FIG. 3A). The efficient recovery of phospho-peptides resulted in increased detection of phosphorylated proteins (FIG. 3B).

In general, phospho-Ser/Thr peptides can be classified as proline-directed or nonproline-directed phosphorylation. Morishima-Kawashima, M. et al. Proline-directed and non-proline-directed phosphorylation of PHF-tau. J. Biol Chem 270, 823-829 (1995). A previous report suggested that proline-directed phospho-peptides may require different β-elimination conditions. Knight, Z. A. et al. Phosphospecific proteolysis for mapping sites of protein phosphorylation. Nat Biotechnol 21, 1047-1054 (2003). To investigate whether our method preferentially isolated proline-directed phospho-peptides, the percentage of proline-directed phospho-peptides presented in the pool of total isolated phospho-peptides was compared. Results showed that the percentage of phospho-(S/T)P peptides were similar between methods using conventional MS and solid-phase enriching MS (FIG. 3C). No apparent reaction preference was evident.

Tau Protein Studies

The longest isoform of human microtubule-associated protein Tau is composed of 441 amino acid residues among which 45 residues are serine and 35 residues are threonine. Mandelkow, E. & Mandelkow, E. M. Microtubules and microtubule-associated proteins. Curr Opin Cell Biol 7, 72-81 (1995). The ability of Tau to facilitate assembly of microtubules is altered by phosphorylation. Alonso, A. C., Zaidi, T., Grundke-Iqbal, I. & Igbal, K. Role of abnormally phosphorylated tau in the breakdown of microtubules in Alzheimer disease. Proc Natl Acad Sci USA 91, 5562-5566 (1994). Tau hyperphosphorylation has been observed in Alzheimer's disease brains (AD), and at least 25 sites on AD Tau are phosphorylated by a variety of serine/threonine kinases. Grundke-Iqbal, I. et al. Abnormal phosphorylation of the microtubule-associated protein tau (tau) in Alzheimer cytoskeletal pathology. Proc Nat! Acad Sci U S A 83, 4913-4917 (1986); Hanger, D. P., Betts, J. C., Loviny, T. L., Blackstock, W. P. & Anderton, B. H. New phosphorylation sites identified in hyperphosphorylated tau (paired helical filament-tau) from Alzheimer's disease brain using nanoelectrospray mass spectrometry. J. Neurochem 71, 2465-2476 (1998); Reynolds, C. H., Betts, J. C., Blackstock, W. P., Nebreda, A. R. & Anderton, B. H. Phosphorylation sites on tau identified by nanoelectrospray mass spectrometry: differences in vitro between the mitogen-activated protein kinases ERK2, c-Jun N-terminal kinase and P38, and glycogen synthase kinase-3beta. J. Neurochem 74, 1587-1595 (2000). This aberrant phosphorylation promotes formation of neurofibrillary tangles. Grundke-Igbal, I. et al. Microtubule-associated protein tau. A component of Alzheimer paired helical filaments. J. Biol Chem 261, 6084-6089 (1986). In addition to AD, tauopathies are also observed in other neurodegenerative disorders. Spillantini, M. G. & Goedert, M. Tau protein pathology in neurodegenerative diseases. Trends Neurosci 21, 428-433 (1998). Currently, research on Tau phosphorylation is restricted to a limited number of phosphorylation-specific antibodies, preventing analysis of global Tau phosphorylation states. Improved detection of phosphorylated sites may provide insight into novel clinical strategies. Previous studies show that aberrant cyclin-dependent kinase 5 (Cdk5) activity may play a critical role in AD pathogensis. Patrick, G. N. et al. Conversion of p35 to p25 deregulates CdkS activity and promotes neurodegeneration. Nature 402, 615-622 (1999); Tseng, H. C., Zhou, Y., Shen, Y. & Tsai, L. H. A survey of CdkS activator p35 and p25 levels in Alzheimer's disease brains. FEBSLett 523, 58-62 (2002). In order to study the Tau phosphoproteome in the context of active Cdk5, a human Tau DNA construct alone or with Cdk5 and Cdk5 activator p25 in neuroblastoma CAD cells was constructed. Heat-stable Tau samples prepared from Tau-transfected CAD cells were trypsinized. Tryptic Tau peptides were then treated with the solid-phase reaction procedure and followed by tandem mass spectrometric analysis. It was found that Thr181, Ser185, Ser191, Ser195, Ser198, Ser199, Ser202, Thr205, Ser208, Ser210, Thr217, Thr220, Ser241, Ser396, and Thr403 were phosphorylated (Table 1). Among these sites, Thr181, Ser199, Ser202, Thr205, Thr217, and Ser396 have (S/T)P motifs representing the consensus Cdk5-phosphorylation sites. The increased phospho-(S/T)P detection could not be explained by methodological bias (FIG. 3C). However, the method had difficulty isolating full length phospho-Tau prior to trypsin digestion. This observation may suggest that the solid-phase procedure is more efficient in capturing phospho-peptides than full-length proteins.

Immunoblots using Tau antibodies showed that phosphorylation of Ser202, Thr205, and Ser404 were significantly increased, whereas no phosphorylation of Thr231 was detected in our Cdk5/p25/Tau sample preparation (FIG. 4A). This result supported the MS results (Table 1).

TABLE 1

| | |
|---|---|
| Transfected with Tau | Transfected with Tau/CDK5/p25 |

| | | |
|---|---|---|
| Tau | ₃₇₁IE [symbol] HKL [symbol] FR₃₇₉ | ₁MADPR₅ |
| | | ₁₆₄GTSNATR₁₇₀ |
| | | ₁₈₁TPPS [symbol] GEPPK₁₉₀ |
| | | ₁₈₁ [symbol] PPS [symbol] GEPPKSGDR₁₉₄ |
| | | ₁₉₁SGDRSGYS [symbol] PGSPGTPGSR₂₀₉ |
| | | ₁₉₁SGDRSGY [symbol] [symbol] PGSPG [symbol] PGSR₂₀₉ |
| | | ₁₉₅SGY [symbol] [symbol] PGSPGTPGSRSR₂₁₁ |
| | | ₂₁₀SRTPSLPTPP [symbol] R₂₂₁ |
| | | ₂₃₅SPSSAK [symbol] R₂₄₂ |
| | | ₃₉₆ [symbol] PVVSGD [symbol] SPR₄₀₆ |
| MAP2 | ₁₄₄₉EPS [symbol] VSR₁₄₅₅ | ₁₅₂₇VSDGVTK₁₅₃₃ |
| | ₁₅₀₆K [symbol] TAAGGESALAPSVFK₁₅₂₂ | ₁₅₂₇V [symbol] DGVTK [symbol] PEK₁₅₂₃₇ |
| | ₁₅₀₇ [symbol] TAAFFESALAPSVFKQAK₁₅₂₅ | ₁₅₅₂RGVSGDR₁₅₅₈ |
| | ₁₅₂₇V [symbol] DGVTK₁₅₃₃ | ₁₆₁₃ [symbol] PGTPGTPSYPR₁₆₂₄ |
| | ₁₅₅₂RGV [symbol] GDR₁₅₅₈ | ₁₇₇₀ARVDHGAEIITQSPGR₁₇₈₅ |
| | ₁₇₆₂LNFREHAK₁₇₆₉ | |

Notes for Table 1.
Peptides derived from over-expressed Tau and endogenous MAP2. Underlining indicates the sites carrying phosphates. Bold font indicates residues carrying the chemical tag. The sequence numberings of Tau and MAP2 peptides are based on the 441 amino acid Tau protein (accession number QRHUT1) and the 1827 amino acid MAP2 protein (accession number NP_002365).

Most phospho-Tau peptides listed in Table 1 were not detected in the protein kinase A-Tau transfected controls (phosphorylation control) or in an unfunctionalized RINK resin control (non-specific binding control) (data not shown). Previous studies have shown that Cdk5 phosphorylation occurs at Thr181, Ser199, Ser202, Thr205, Thr212, Thr217, Thr231, Ser235, Ser396 and Ser404. Alonso, A. C., Zaidi, T., Grundke-Iqbal, I. & Igbal, K. Role of abnormally phosphorylated tau in the breakdown of microtubules in Alzheimer disease. Proc Natl Acad Sci USA 91, 5562-5566 (1994); Grundke-Iqbal, I. et al. Abnormal phosphorylation of the microtubule-associated protein tau (tau) in Alzheimer cytoskeletal pathology. Proc Nat. Acad Sci U S A 83, 4913-4917 (1986). Recently, Cruz et al studied the phosphorylation of endogenous Tau in a p25-overexpressing mouse forebrain using conventional tandem mass spectrometry. Cruz, J. C., Tseng, H.-C., Goldman, J. A., Shih, H. & Tsai, L.-H. Aberrant Cdk5 activation by p25 triggers pathological events leading to neurodegeneration and neurofibrillary tangles. Neuron 40, 471-483 (2003). In that report, phosphorylation of Ser199, Ser202, Thr205, Thr217, Thr220, Ser396, and Thr403 was reported. Thus, results shown in Table 1 agree with those of previous reports. Hanger, D. P., Betts, J. C., Loviny, T. L., Blackstock, W. P. & Anderton, B. H. New phosphorylation sites identified in hyperphosphorylated tau (paired helical filament-tau) from Alzheimer's disease brain using nanoelectrospray mass spectrometry. J. Neurochem 71, 2465-2476 (1998); Reynolds; C. H., Betts, J. C., Blackstock, W. P., Nebreda, A. R. & Anderton, B. H. Phosphorylation sites on tau identified by nanoelectrospray mass spectrometry: differences in vitro between the mitogen-activated protein kinases ERK2, c-Jun Nterminal kinase and P38, and glycogen synthase kinase-3β. J. Neurochem 74, 1587-1595 (2000); Cruz, J. C., Tseng, H.-C., Goldman, J. A., Shih, H. & Tsai, L.-H. Aberrant Cdk5 activation by p25 triggers pathological events leading to neurodegeneration and neurofibrillary tangles. Neuron 40, 471-483 (2003).

To investigate whether the chemical method was applicable to endogenous proteins, the phosphorylation states of endogenous microtubule-associated protein 2 (MAP2) in the CAD cells were examined. MAP2 and Tau share nearly identical microtubulebinding motif repeats. Goedert, M., Crowther, RCA. & Garner, C. C. Molecular characterization of microtubule-associated proteins tau and MAP2. Trends Neurosci 14, 193-199 (1991). Like Tau, MAP2 is also phosphorylated by multiple kinases. Sanchez, C., Diaz-Nido, J. & Avila, J. Phosphorylation of microtubule-associated protein 2 (MAP2) and its relevance for the regulation of the neuronal cytoskeleton function. Prog Neurobiol 61, 133-168 (2000). It was found that MAP2 was hyperphosphorylated in cells transfected with Tau (Table 1). This is consistent with a previous study reporting that MAP2 was constitutively hyperphosphorylated. Tsuyama, S., Terayama, Y. & Matsuyama, S. Numerous phosphates of microtubule-associated protein 2 in living rat brain. J. Biol Chem 262, 10886-10892 (1987). However, in p35/Cdk5-expressing cells, it was found that phosphorylation of MAP2 was limited to the (S/T)P sites (Table 1). Differences in phosphorylation between MAP2 and Tau appeared primarily in the low homology regions (FIG. 4B). Ser202/Thr2O5 and Ser396, the two main Cdk5 phosphorylation modules in Tau, are also present at analogous regions of MAP2 (FIG. 4B). Thus, the regions of 1613TPGTPGTP1620 on MAP2 and 199SPGSPGTP206 on Tau may represent an ideal Cdk5 phosphorylation motif, (S/T)PG(S/T)PGTP. This notion was confirmed by using active Cdk5 to phosphorylate (S/T)PG(S/T)PGTP peptides in vitro (data not shown). Moreover, this motif features two consecutive PXXP sequences which represent SH3 domain binding sites. Lee, G., Newman, S. T., Gard, D. L., Band, H. & Panchamoorthy, G. Tau interacts with src-family non-receptor tyrosine kinases. J Cell Sci 111 (Pt 21), 3167-3177 (1998); Lim, R. W. & Halpain, S. Regulated association of microtubule-associated protein 2 (MAP2) with Src and Grb2: evidence for MAP2 as a scaffolding protein. JBiol Chem 275, 20578-20587 (2000). The motif is also part of the proline-rich region of Tau important for binding of WW domain-containing protein Pin1. Studies are in progress to determine the biological consequences of these phosphorylation events.

Synopsis

Techniques have been developed for mass-spectrometry-based phosphorylation determination, including phospho-specific antibodies, affinity chromatography, chemical modification, precursor ion scanning, neutral-loss scanning, and electron capture dissociation technique. Mann, M. et al. Analysis of protein phosphorylation using mass spectrometry: deciphering the phosphoproteome. Trends Biotechnol 20, 261-268 (2002); Knight, Z. A. et al. Phosphospecific proteolysis for mapping sites of protein phosphorylation. Nat Biotechnol 21, 1047-1054 (2003); Ficarro, S. B. et al. Phosphoproteome analysis by mass spectrometry and its application to Saccharomyces cerevisiae. Nat Biotechnol 20, 301-305 (2002). For experiment-driven phosphoproteomic studies, a simple, efficient, and comprehensive method is necessary. From a previous report, the feasibility of applying (β-elimination/Michael addition for phospho-peptide analysis was questionable. Oda, Y., Nagasu, T. & Chait, B. T. Enrichment analysis of phosphorylated proteins as a tool for probing the phosphoproteome. Nat Biotechnol 19, 379-382. (2001). However, it has been found that the presently disclosed solid-phase procedure was able to efficiently isolate phospho-peptides based on the results presented. This was demonstrated by the significant improvement in efficiency over conventional and neutralloss of phosphoric acid mass spectrometry. There are several possible, non-binding, explanations for the increased efficiency. First, the decreased number of operating steps may result in the increased sensitivity of (β-elimination as compared to previous procedures. Meyer, H. E., Hoffmann-Posorske, E. & Heilmeyer, L. M., Jr. Determination and location of phosphoserine in proteins and peptides by conversion to Sethylcysteine. Methods Enzymol 201, 169-185 (1991); Zhou, H., Watts, J. D. & Aebersold, R. A systematic approach to the analysis of protein phosphorylation. Nat Biotechnol 19, 375-378. (2001). Second, labeling of phosphorylation sites and enrichment of phospho-peptides occur in a single solid-phase step. Third, the mass size of the 6-(2-mercapto-acetylamine)-hexanoic amide tag, which is smaller than a previously described biotin tag^{s}, allows for a wider detection window in mass spectrometric measurement. Fourth, barium hydroxide catalyzes βelimination more rapidly than sodium hydroxide or potassium hydroxide, and is most efficient for our procedure. Patrick, G. N. et al. Conversion of p35 to p25 deregulates CdkS activity and promotes neurodegeneration. Nature 402, 615-622. (1999). Beta-elimination may also occur on carbamidomethylated cysteinyl or O-linked glycosylated peptides. Byford, M. F. Rapid and selective modification of phosphoserine residues catalysed by Ba2+ ions for their detection during peptide microsequencing. Biochem J 280 (Pt 1), 261-265 (1991). Oxidation of cysteinyl residues is an alternative used to modify cysteine residues. Mann, M. et al. Analysis of protein phosphorylation using mass spectrometry: deciphering the phosphoproteome. Trends Biotechnol 20, 261-268 (2002). However, when using barium hydroxide as the alkaline source, reaction rates of β-elimination of O-linked glycosylated peptides are two orders of magnitude slower compared to those of phospho-Ser/Thr peptides. Patrick, G. N. et al. Conversion of p35 to p25 deregulates CdkS activity and promotes neurodegeneration. Nature 402, 615-622. (1999). Based on the results presented herein, it is concluded that the combination of (β-elimination and solid-phase Michael addition is capable of capturing, releasing, and tagging complex phospho-Ser/Thr peptide mixtures.

Definitions

For convenience, certain terms employed in the specification, examples and appended claims are collected here. These definitions should be read in light of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used herein to mean “including but not limited to”. “Including” and “including but not limited to” are used interchangeably.

The term “label” refers to a composition detectable by spectroscopic, photochemical, biochemical, immunochemical chemical or other means. For example, useful labels include deuterium, ³²P, chromophores, fluorophores, fluorescent proteins, electron-dense reagents, enzymes (e.g., as commonly used in an ELISA), biotin, dioxigenin, or haptens and proteins for which antisera or monoclonal antibodies are available. A label often generates a measurable signal, such as radioactivity, fluorescent light or enzyme activity, which may in certain instances be used to quantitate the amount of label present.

The term “labeled” refers to incorporation of a detectable marker into a molecule, such as a polypeptide, small molecule or subject composition. Various methods of labeling polypeptides and other molecules are known in the art and may be used. Examples of labels include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance and other complications.

The terms “Lewis acid” and “Lewis acidic” are art-recognized and refer to chemical moieties which can accept a pair of electrons from a Lewis base as defined above.

The terms “Lewis base” and “Lewis basic” are art-recognized and generally refer to a chemical moiety capable of donating a pair of electrons under certain reaction conditions. It may be possible to characterize a Lewis base as donating a single electron in certain complexes, depending on the identity of the Lewis base and the metal ion, but for most purposes, however, a Lewis base is best understood as a two electron donor. Examples of Lewis basic moieties include uncharged compounds such as alcohols, thiols, and amines, and charged moieties such as alkoxides, thiolates, carbanions, and a variety of other organic anions. In certain examples, a Lewis base may consist of a single atom, such as oxide (O²⁻). In certain, less common circumstances, a Lewis base or ligand may be positively charged. A Lewis base, when coordinated to a metal ion, is often referred to as a ligand. Further description of ligands relevant to the present invention is presented herein.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “complex” is art-recognized and refers to a compound formed by the union of one or more electron-rich and electron-poor molecules or atoms capable of independent existence with one or more electronically poor molecules or atoms, each of which is also capable of independent existence. Examples of complexes include associations between antigen/antibodies, lectin/avidin, target polynucleotide/probe oligonucleotide, antibody/anti-antibody, receptor/ligand, enzyme/ligand, polypeptide/polypeptide, polypeptide/polynucleotide, polypeptide/co-factor, polypeptide/substrate, polypeptide/inhibitor, polypeptide/small molecule, and the like. “Member of a complex” refers to one moiety of the complex, such as an antigen or ligand. “Protein complex” or “polypeptide complex” refers to a complex comprising at least one polypeptide.

The terms “combinatorial library” or “library” are art-recognized and refer to a plurality of compounds, which may be termed “members,” synthesized or otherwise prepared from one or more starting materials by employing either the same or different reactants or reaction conditions at each reaction in the library. There are a number of other terms of relevance to combinatorial libraries (as well as other technologies). The term “identifier tag” is art-recognized and refers to a means for recording a step in a series of reactions used in the synthesis of a chemical library. The term “immobilized” is art-recognized and, when used with respect to a species, refers to a condition in which the species is attached to a surface with an attractive force stronger than attractive forces that are present in the intended environment of use of the surface, and that act on the species. The term “solid support” is art-recognized and refers to a material which is an insoluble matrix, and may (optionally) have a rigid or semi-rigid surface. The term “linker” is art-recognized and refers to a molecule or group of molecules connecting a support, including a solid support or polymeric support, and a combinatorial library member. The term “polymeric support” is art-recognized and refers to a soluble or insoluble polymer to which a chemical moiety can be covalently bonded by reaction with a functional group of the polymeric support. The term “functional group of a polymeric support” is art-recognized and refers to a chemical moiety of a polymeric support that can react with an chemical moiety to form a polymer-supported amino ester.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “structure-activity relationship” or “(SAR)” is art-recognized and refers to the way in which altering the molecular structure of a drug or other compound or coordination complex alters its interaction with a receptor, enzyme, nucleic acid or other target and the like.

The term “agonist” is art-recognized and refers to a compound or coordination complex that mimics the action of natural transmitter or, when the natural transmitter is not known, causes changes at the receptor complex in the absence of other receptor ligands.

The term “antagonist” is art-recognized and refers to a compound or coordination complex that binds to a receptor site, but does not cause any physiological changes unless another receptor ligand is present.

The term “competitive antagonist” is art-recognized and refers to a compound or coordination complex that binds to a receptor site; its effects may be overcome by increased concentration of the agonist.

The term “partial agonist” is art-recognized and refers to a compound or coordination complex that binds to a receptor site but does not produce the maximal effect regardless of its concentration.

The term “aliphatic” is art-recognized and refers to a linear, branched, cyclic alkane, alkene, or alkyne. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl(alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles,” heteroaromatics,” or “heteroaryl.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 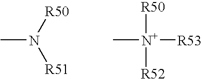
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “amine” is art-recognized are refers to a compound containing an ammonia moiety or moieties coordinated to a metal ion. The term “ammonia” is art-recognized an refers to an amine group substituted with hydrogens.

The term “acylamino” is art-recognized and refers to a moiety that may be represented by the general formula: 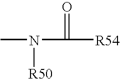
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 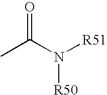
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 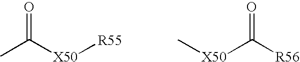
wherein X50 is a bond or represents an oxygen or a sulfur, and R55 and R56 represents a hydrogen, an alkyl, an alkenyl, —(CH₂)ₘ—R61or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 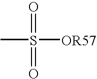
in which R57 is an electron pair, hydrogen, alkyl, cycloalkyl, or aryl.

The term “sulfate” is art recognized and includes a moiety that may be represented by the general formula: 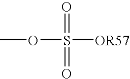
in which R57 is as defined above.

The term “sulfonamido” is art recognized and includes a moiety that may be represented by the general formula: 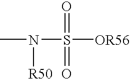
in which R50 and R56 are as defined above.

The term “sulfamoyl” is art-recognized and refers to a moiety that may be represented by the general formula: 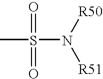
in which R50 and R51 are as defined above.

The term “sulfonyl” is art-recognized and refers to a moiety that may be represented by the general formula: 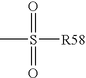
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 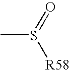
in which R58 is defined above.

The term “phosphoryl” is art-recognized and may in general be represented by the formula: 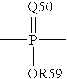
wherein Q50 represents S or O, and R59 represents hydrogen, a lower alkyl or an aryl. When used to substitute, e.g., an alkyl, the phosphoryl group of the phosphorylalkyl may be represented by the general formulas: 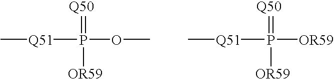
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 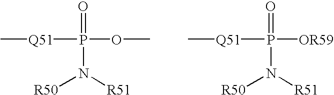
wherein Q51, R50, R51 and R59 are as defined above.

The term “phosphonamidite” is art-recognized and may be represented in the general formulas: 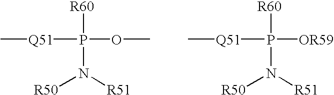
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds of the present invention may exist in particular geometric or stereoisomeric forms. In addition, polymers of the present invention may also be optically active. The present invention contemplates all such compounds, including cis- and trans-isomers, R— and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (3^{rd} ed., Wiley: New York, 1997).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “amino acid” is art-recognized and refers to all compounds, whether natural or synthetic, which include both an amino functionality and an acid functionality, including amino acid analogs and derivatives.

The terms “amino acid residue” and “peptide residue” are art-recognized and refer to an amino acid or peptide molecule without the —OH of its carboxyl group.

The term “amino acid residue” further includes analogs, derivatives and congeners of any specific amino acid referred to herein, as well as C-terminal or N-terminal protected amino acid derivatives (e.g. modified with an N-terminal or C-terminal protecting group).

The names of the natural amino acids are abbreviated herein in accordance with the recommendations of IUPAC-IUB.

A “reversed” or “retro” peptide sequence as disclosed herein refers to that part of an overall sequence of covalently-bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman et al. Accounts of Chem. Res. 12:423 (1979).

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention may be formed by utilizing an appropriate amino acid mimetic moiety to link the two adjacent portions of the sequences depicted above utilizing a reversed peptide (reversed amide) bond.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is usually (D), and the configuration of the non-reversed portion is usually (L). Opposite or mixed configurations are acceptable when appropriate to optimize a binding activity.

The term “nucleic acid” is art-recognized and refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single-stranded (such as sense or antisense) and double-stranded polynucleotides.

The terms “gene” or “recombinant gene” are art-recognized and refer to a nucleic acid comprising an open reading frame encoding a polypeptide, including both exonic and (optionally) intronic sequences.

The term “gene construct” is art-recognized and refers to a vector, plasmid, viral genome or the like which includes an “coding sequence” for a polypeptide or which is otherwise transcribable to a biologically active RNA (e.g., antisense, decoy, ribozyme, etc), can transfect cells, in certain embodiments mammalian cells, and may cause expression of the coding sequence in cells transfected with the construct.

The term “homology” is art-recognized and refers to sequence similarity between two peptides or between two nucleic acid molecules.

The term “operably linked” is art-recognized and refers to the relationship between two nucleic acid regions, means that they are functionally related to each other.

The term “antisense” nucleic acid is art-recognized and refers to oligonucleotides which specifically hybridize (e.g., bind) under cellular conditions with a gene sequence, such as at the cellular mRNA and/or genomic DNA level, so as to inhibit expression of that gene, e.g., by inhibiting transcription and/or translation. The binding may be by conventional base pair complementarily, or, for example, in the case of binding to DNA duplexes, through specific interactions in the major groove of the double helix.

The term “host cell” is art-recognized and refers to a cell transduced with a specified transfer vector. The cell is optionally selected from in vitro cells such as those derived from cell culture, ex vivo cells, such as those derived from an organism, and in vivo cells, such as those in an organism. “Recombinant host cells” refers to cells which have been transformed or transfected with vectors constructed using recombinant DNA techniques.

The terms “recombinant protein,” “heterologous protein” and “exogenous protein” are art-recognized and are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

The term “regulatory element” is art-recognized and refers to nucleotide sequences (such as DNA sequences) that induce or control transcription of protein coding sequences with which they are operably linked. Examples of regulatory elements categorized by function include initiation signals, enhancers, promoters and the like. Exemplary regulatory elements are described in Goeddel; Methods in Enzymology 185 (1990). In certain embodiments, transcription of a gene or other DNA is under the control of a promoter sequence (or other regulatory element) which controls the expression of a coding sequence in a cell-type in which expression is intended. A variety of promoters categorized by function are known. The term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of a urogenital origin, e.g., renal cells, or cells of a neural origin, e.g., neuronal cells. The term also covers so-called “leaky” promoters, which regulate expression of a selected DNA primarily in one tissue, but cause expression in other tissues as well. The term “inducible” promoter refers to a promoter which is under environmental or developmental regulation. The term “constitutive” promoter refers to a promoter which is active under most environmental and developmental conditions.

The term “transfection” is art-recognized and refers to the introduction of a nucleic acid, e.g., an expression vector, into a recipient cell, which in certain embodiments may be by nucleic acid-mediated gene transfer. “Transformation,” as used with respect to transfected nucleic acid, is an art-recognized term and refers to a process in which a cell's genotype is changed as a result of the cellular uptake of exogenous nucleic acid.

The term “transfer vector” is art-recognized and refers to a first nucleic acid molecule to which a second nucleic acid has been linked, and includes for example plasmids, cosmids or phages (as discussed in grater detail below). In certain embodiments of the present invention, the therapeutic agent is the second nucleic acid. One type of transfer vector is an episome, i.e., a nucleic acid capable of extra-chromosomal replication.

In certain embodiments, a transfer vector may be an “expression vector,” which refers to a replicable DNA construct used to express DNA which encodes the desired protein and which includes a transcriptional unit comprising an assembly of (i) genetic element(s) having a regulatory role in gene expression, for example, promoters, operators, or enhancers, operatively linked to (ii) a DNA sequence encoding a desired protein which is transcribed into mRNA and translated into protein, and (iii) appropriate transcription and translation initiation and termination sequences. In certain embodiments, the therapeutic agent is the DNA sequence. The choice of promoter and other regulatory elements generally varies according to the intended host cell. In general, expression vectors of utility in recombinant DNA techniques are often in the form of “plasmids,” which refer to circular double stranded DNA loops which, in their vector form are not bound to the chromosome. The invention is intended to include such other forms of expression vectors which serve equivalent functions and which become known in the art subsequently hereto.

Certain transfer vectors may contain regulatory elements for controlling transcription or translation, which may be generally derived from mammalian, microbial, viral or insect genes. The ability to replicate in a host, usually conferred by an origin of replication, and a selection gene to facilitate recognition of transformants, may additionally be incorporated.

The design of any transfer vector may depend on such factors as the choice of the host cell to be transformed and/or the type of protein desired to be expressed. Moreover, the vector's copy number, the ability to control that copy number and the expression of any other proteins encoded by the vector, such as antibiotic markers (e.g., ampicillin), may also be considered.

The term “transgenic animal” is art-recognized and refers to any animal, often a non-human mammal, a bird or an amphibian, in which one or more of the cells of the animal contain nucleic acid introduced by way of human intervention, such as by transgenic techniques well known in the art. Such nucleic acid may be referred to as a “transgene.” The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. A transgene may be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene may also be present in a cell in the form of an episome. A transgene may include one or more regulatory elements and any other nucleic acid, such as introns, that may be necessary for optimal expression of a selected nucleic acid. In certain embodiments, a transgene comprises a nucleic acid sequence of interest and one or more regulatory elements for controlling transcription of the nucleotide sequence encoded by such nucleic acid sequence, e.g., the regulatory element is operably linked to a nucleic acid.

In certain embodiments, the transgene or other therapeutic agent may be a “gene therapy construct,” which is an expression vector which may alter the phenotype of a cell when taken up by the cell, or a gene construct. In certain embodiments, the gene therapy construct may be a “recombinant coding sequence” which encodes a polypeptide, or is transcribable to an antisense nucleic acid, a ribozyme, or any other RNA product which alters the phenotype of the cell in which it is produced. “Recombinant gene” refers to a genetic construct including a “recombinant coding sequence.”

The term “antibody” is art-recognized and refers to whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal or other purified preparations of antibodies and recombinant antibodies.

“Human monoclonal antibodies” or “humanized” murine antibodies, as the terms are used herein, refer to murine monoclonal antibodies “humanized” by genetically recombining the nucleotide sequence encoding the murine Fv region (i.e., containing the antigen binding site) or the complementarity-determining regions thereof with the nucleotide sequence encoding at least a human constant domain region and an Fc region, e.g., in a manner similar to that disclosed in European Patent Application Publication No. 0 411 893 A3. Some additional murine residues may also be retained within the human variable region framework domains to ensure proper target site binding characteristics. In certain embodiments, humanized antibodies may decrease the immunoreactivity of the antibody or polypeptide in the host recipient, permitting an increase in the half-life and a reduction in the possibility of adverse immune reactions.

An “imaging agent” shall mean a composition capable of generating a detectable image upon binding with a target and shall include radionuclides (e.g., In-111, Tc-99m, I-123, I-125 F-18, Ga-67, Ga-680); for Positron Emission Tomography (PET) and Single Photon Emission Tomography (SPECT), unpair spin atoms and free radicals (e.g., Fe, lanthanides, and Gd); and contrast agents (e.g., chelated (DTPA) manganese) for Magnetic Resonance Imaging (MRI). Imaging agents are discussed in greater detail below.

The term “small molecule” is art-recognized and refers to a composition which has a molecular weight of less than about 2000 amu, or less than about 1000 amu, and even less than about 500 amu. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

A “target” shall mean a site to which targeted constructs bind. A target may be either in vivo or in vitro. In certain embodiments, a target may be a tumor (e.g., tumors of the brain, lung (small cell and non-small cell), ovary, prostate, breast and colon as well as other carcinomas and sarcomas). In other embodiments, a target may be a site of infection (e.g., by bacteria, viruses (e.g., HIV, herpes, hepatitis) and pathogenic fungi (Candida sp.). In still other embodiments, a target may refer to a molecular structure to which a targeting moiety binds, such as a hapten, epitope, receptor, dsDNA fragment, carbohydrate or enzyme. Additionally, a target may be a type of tissue, e.g., neuronal tissue, intestinal tissue, pancreatic tissue etc, or any cell type.

The term “targeting moiety” refers to any molecular structure which assists a compound or other molecule in binding or otherwise localizing to a particular target, a target area, entering target cell(s), binding to a target receptor, etc. For example, targeting moieties may include peptides, lipids (including cationic, neutral, and steroidal lipids, virosomes, and liposomes), antibodies, lectins, ligands, sugars, steroids, hormones, nutrients, and proteins may serve as targeting moieties. Other examples of targeting moieties are described in more detail below.

The term “isolated”, as used herein with reference to proteins and other biological materials, refers to a preparation of protein or material that is essentially free from contaminating proteins and other materials that normally would be present in association with the protein or material, e.g., in the cellular milieu in which the protein or complex is found endogenously. Thus, an isolated protein is isolated from cellular components that normally would “contaminate” or interfere with the study of the protein in isolation, for instance while screening for inhibitors thereof. It is to be understood, however, that an “isolated” complex may incorporate other proteins the modulation of which, by the subject protein or protein complex, is being investigated.

The term “interact” as used herein is meant to include detectable relationships or associations (e.g. biochemical interactions) between molecules, such as interactions between protein-protein, protein-nucleic acid, nucleic acid-nucleic acid and protein-small molecule or nucleic acid-small molecule in nature.

The term “modulation” is art-recognized and refers to up regulation (i.e., activation or stimulation), down regulation (i.e., inhibition or suppression) of a response, or the two in combination or apart.

The term “treating” is art-recognized and refers to curing as well as ameliorating at least one symptom of any condition or disease.

The term “prophylactic” or “therapeutic” treatment is art-recognized and refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

A “patient,” “subject” or “host” to be treated by the subject method may mean either a human or non-human animal.

The term “mammal” is known in the art, and exemplary mammals include humans, primates, bovines, porcines, canines, felines, and rodents (e.g., mice and rats).

The term “bioavailable” is art-recognized and refers to a form of the subject invention that allows for it, or a portion of the amount administered, to be absorbed by, incorporated to, or otherwise physiologically available to a subject or patient to whom it is administered.

The term “pharmaceutically-acceptable salts” is art-recognized and refers to the relatively non-toxic, inorganic and organic acid addition salts of compounds, including, for example, coordination complexes of the present invention.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject supplement, composition, therapeutic or other material other than directly into the central nervous system, such that it enters the patient's system and, thus, is subject to metabolism and other like processes, for example, subcutaneous administration.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

Kits

The invention also provides kits containing reagents or compositions of the invention. Thus, the invention provides a kit with a solid-phase capture and tag reagent, exemplified as in FIG. 1. The contents of the kit of the invention, for example, a solid-phase capture and tag reagent or composition of the invention, are contained in packaging material, and, if desired, a sterile, contaminant-free environment. In addition, the packaging material contains instructions indicating how the materials within the kit can be employed to label sample molecules. The instructions for use typically include a tangible expression describing the reagent concentration or at least one assay method parameter, such as the relative amounts of reagent and sample to be admixed, maintenance time periods for reagent/sample admixtures, temperature, buffer conditions, and the like.

### Exemplification

Materials—RINK resin (ArgoGel-RINK-NH-Fmoc) was purchased from Argonaut Technologies (San Carlos, Calif.). PyBOP (benzotriazole-1-yl-oxy-tris-pyrrolidino-phosphonium hexafluorophosphate) and SAMA-OPfp (S-acetylthioglycolic acid pentafluorophenyl ester) were purchased from Calbiochem-Novabiochem AG (Laufelfingen, Switzerland). Synthetic phospho-Ser CREB peptide was obtained from New England Biolabs. Phospho-Thr VDAAV(phospho-T)TEERHC peptide was synthesized at Tufts Core Facility, Tufts University, Medford, Mass. Phos-Select™ iron affinity gel, an iron(III)-nitriloacetic acid gel, and MAP2 protein were obtained from Sigma-Aldrich (St. Louis, Mo.). PhosphoThr amyloid precursor protein peptide was synthesized at Tufts Core Facility, Tufts University, Medford, Mass. Tau-5 (monoclonal) and Tau [pThr23 1] (polyclonal) antibodies were purchased from Biosource (Camarillo, Calif.); AT-8 (monoclonal) from Innogentics (Belgium). PHF-1 (monoclonal) antibody is a gift from M.D. Nguyen (Harvard Medical School). Horseradish peroxidase (HRP)-conjugated AntiMouse/AntiRabbit IgG secondary antibodies were purchased from Amersham Biosciences (Piscataway, N.J.). Chemiluminescence reagents used in immunoblotting assay were purchased from NEN Life Science Products (Botson, Mass.). All other compounds are reagent grade. The DNA constructs hTau4O, Cdk5-HA, and p25-HA were described previously. Patrick, G. N. et al. Conversion of p35 to p25 deregulates CdkS activity and promotes neurodegeneration. Nature 402, 615-622. (1999). Active CDK5/p25 complex was provided by Andrea Musacchio at European Institute of Oncology, Italy.

Mass spectrometry—MALDI-TOF mass spectrometric analysis of synthetic peptides was performed in a Voyger DE-STR MALDI-TOF mass spectrometer (Applied Biosystems, Foster City, Calif.) in the positive ion, delay-extraction, and reflector-on mode. General settings of the MALDI-TOF experiments were as follows: accelerating voltage 20,000 V, grid voltage 75%, guide wire 0.02%, extraction delay time 200 nano-sec. Peptide samples were desalted with ZipTip C 18 preloaded tips (Millipore, Bedford, Mass.). Bound peptides were eluted with 2 μL of a solution containing 10 mg/mL a-cyan-4-hydroxycinnamic acid, 50% actetonitril, 10% ethanol, and 0.1% TFA solution and spotted directly onto a gold-coated target plate. An LCZ LC-MS instrument (Water, Milford, Mass.) was used to monitor the synthesis of the resin by analyzing acid-cleaved products. For the analysis of microtubuleassociated proteins, a Micro Q-TOF system was used (Waters, Milford, Mass.). The system includes a HPLC liquid chromatography (CapLC) online-coupled with a nanoelectrospray ionization quadrupole-time of flight instrument. Cleaved products derived from a starting material of 500 μg peptide samples (Tau, Cdk5-Tau, and PKA-Tau) or 250 μg heat-stable samples were injected into the HPLC system equipped with a PicoFrit C18 column (New Objective, Woburn, Mass.). Mixtures of peptides were resolved in a 90-minute gradient (5-40% of acetonitril) with a flow rate of 0.8 μl/min. Top three strongest ion peaks in parent-ion-scan mode (m/z 400-2000, 1 sec/scan) were automatically selected for subsequent CID analysis (m/z 90-2000, 1.5 sec/scan). Collision energy in CID analysis used a default mass-charge dependent profile provided by the vender. For phospho-peptide detection of synaptosomal membrane proteins, 10 μg of peptides (synaptosomal peptides) were injected to the HPLC system equipped with a PicoFrit C18 column (New Objective, Woburn, Mass.). TFA cleaved tagged peptides which were derived from a starting material of 10 μg peptides were also analyzed with the same system. Mixtures of peptides were resolved in a 120 minute gradient (5-40% of acetonitril) with a flow rate of 0.8 μl/min. The top three strongest ion peaks in parent-ion-scan mode (m/z 400-1800, 1 sec/scan) were automatically selected for subsequent CID analysis (m/z 90-1800, 1.5 sec/scan). Collision energy in CID analysis used a default mass-charge-dependent profile provided by the vender. In neutral-loss of phosphoric acid mass spectrometric analysis, the parent survey scan used 30 voltage for high collision energy, and 5 for low collision energy. Neutral loss was set to 97.9940, 195.9538, and 293.9307 Daltons for single and multiple loss of phosphoric acid. The ion-trap tandem mass spectrometry was carried out at WEMB Biochem. Inc. (Toronto, Canada). The mass spectrometer LCQ Deca XP was coupled to a micro-spray Vydac C18 column HPLC system and operated by Xcalibur 1.2 XP1 software.

Analysis of mass spectrometric data—Raw data obtained from Q-TOF tandem mass spectrometric analysis was processed with MassLynx 4.0 software (Waters, Milford, Mass.) to generate peak lists. Raw data acquired from ion-trap tandem mass spectrometric analysis was processed with Biowork 3.1 software (Thermo Electron, Calif.) to generate peak lists. Peak lists of each sample were submitted to a Mascot server (Matrixscience, London, UK) and searched against the SwissProt protein database. Unmatched peak lists were further submitted to the Mascot server against a customized MAP-Tau database. Tolerance of parent ions and daughter ions were 1.5 and 0.6 Daltons, respectively. All Mascot results were also cross-examined with their corresponding CID spectra manually to ensure fidelity of the computational outcome.

### EXAMPLE 1

Synthesis of Nucleophilic RINK Resin

Procedure 1

Synthesis of beads is basically based on Fmoc chemistry. Coupling efficiencies were monitored by the Kaiser test. SPPS was carried out on an 180° Variable Rate Shaker (Peptides International, Louisville, Ky.). The Fmoc group on AgroGel RINK-NH-Fmoc was removed in 20% piperidine/DMF solution. Beads were washed extensively in DMF (N,N-dimethylformamide, American Bioanalytical, Natic, Mass.). Fmoc-E-Ahx-OH (Nfluorenyl oxycarbonyl hexanoic acid, 5 equivalents) or D8 isotopically labeled Fmoc-sAhx-OH was pre-activated with PyBOP (6 equivalents) and DIPEA (diisopropylethylamine, 6 equivalents) in DMF for 2 minutes and added to the resin. After 30 minutes, the resin was washed extensively and followed by a second coupling overnight and a standard Fmoc removal procedure. SAMA-OPfp (5 equivalents) was added in DMF and resin was incubated for 30 minutes followed by a second coupling overnight. The acetyl group which prevented the thiol group from oxidation during storage was removed several hours before use of the resin in DMF containing 10% ammonium hydroxide for 1 hour followed by extensive washing with DMF (3×), ethanol (3×) and water (3×). Coupling efficiencies were monitored by the Kaiser test.

Procedure 2

Synthesis of the resin is based on Fmoc chemistry to add the 6-(mercapto-acetylamino)-hexanoic acid group onto AgroGel RPNK-NH-Fmoc resin (approximate ligand stoichiometry, 0.3 mmol/gram of resin). Solid-phase peptide synthesis was carried out on an 180° Variable Rate Shaker (Peptides International, Louisville, Ky.). 2.5 grams (dry weight) of AgroGel RINK-NH-Fmoc resin were first swelled in 20 mL of DMF solvent for 10 minutes at room temperature and then incubated in 50 mL of 20% piperidine/DMF solution for 1 hour at room temperature in order to remove the Fmoc group. After Fmoc removal, resins were washed extensively in DMF (N,N-dimethylformamide, American Bioanalytical, Natic, Mass.). Fmoc-ε-Ahx-OH (N-fluorenyl oxycarbonyl hexanoic acid, 5 equivalents to the number of RINK-NH-Fmoc moles on the resins) was pre-activated with PyBOP (6 equivalents to the number of Fmoc-ε-Ahx-OH moles) and DIPEA (di-isopropylethylamine, 6 equivalents to the number of Fmoc-ε-Ahx-OH moles) in DMF for 2 minutes and added to the resin. After 30 minutes, the resin was washed extensively and followed by a second coupling overnight. A standard Fmoc removal procedure of the protective group was applied to remove the terminal Fmoc group. SAMA-OPfp (5 equivalents to the number of RINK-NH-Fmoc moles) was added in DMF and the resin was incubated for 30 minutes followed by a second coupling overnight. The terminal acetyl group on the ligand, which prevents the thiol group from oxidation during storage, was removed before use of the resin in DMF containing 10% ammonium hydroxide for 1 hour followed by extensive washing with DMF (3 times), ethanol (3 times) and water (3 times). Completion of each coupling step was monitored by the Kaiser test which detected free terminal amino groups in solid-phase synthesis of peptides[44]. An LCT LC-MS instrument (Water, Milford, Mass.) was used to monitor the synthesis of the resin by analyzing acid-cleaved products.

### EXAMPLE 2

β-Elimination and Solid-Phase Michael Addition

Procedure 1

One-pot (3-elimination and Michael addition reactions were effected in 20 mM Ba(OH)₂ and 10% acetonitrile at 37° C. under gentle agitation for 16 hours. Freshly prepared 2-mercaptoacetyl-hexanoyl functionalized beads (30 μL of bed volume) were used in a reaction with 1 pmol of synthetic polypeptides or with 500 μg of cellular polypeptides. Unfunctionalized RINK resin was used in parallel as a control to examine non-specific binding. Reactions were terminated by adjusting pH to 5 with acetic acid. Reaction solutions were removed and the resins were collected after a brief centrifugation. Resins were washed subsequently with PBS (phosphate-buffered saline), 50% methanol, 90% acetonitril, 100% DMF, and 100% methanol, with two washes of each solvent for five minutes. Resins were dried in a SpeedVac. Peptide cleavage was carried out by addition of 80% TFA (trifluoroacetic acid) to the resins for 30 minutes at room temperature, followed by another 30-minute cleavage with fresh 80% TFA. Peptides were precipitated in cold diethyl ether and collected after centrifugation at 15,000×g for 10 minutes. The isolated peptides were dried in a SpeedVac and used for mass spectrometric analysis.

Procedure 2

One-pot β-elimination and Michael addition reactions were effected in 10% acetonitrile and 20 mM Ba(OH)₂, pH 12.75 in closed microcentrifuge tubes at 37° C. under gentle agitation for 16 hours. Freshly prepared 2-mercaptoacetyl-hexanoyl functionalized beads (30 μL of bed volume) were used in a reaction with 1 pmol of synthetic polypeptides or with 500 μg of cellular polypeptides. Unfunctionalized RINK resin was used in parallel as a control to examine non-specific binding. Reactions were terminated by adjusting the pH to 5 with acetic acid. Reaction solutions were removed and the resins were collected after brief centrifugation. Resins were subsequently washed in sequence with PBS (phosphate-buffered saline), 50% methanol, 90% acetonitrile, 100% DMF, and 100% methanol, with two washes in each solvent for five minutes. Resins were dried in a SpeedVac. Peptide cleavage was carried out by two steps of incubating 200 μL of 20% TFA (trifluoroacetic acid in water) to the resins at room temperature for 30 min each. A total of 400 μL of cleavage products was collected and the volume was reduced to 100 μL in a SpeedVac. Peptides in the TFA solution were precipitated in 1 mL of cold diethyl ether and collected after centrifugation at 15,000×g for 10 minutes. The isolated peptides were dried in a SpeedVac and used for mass spectrometric analysis.

### EXAMPLE 3

Synaptosomal Preparation—The preparation of mouse brain synaptosomes was adapted and modified from a method described by Li et al. Li, X. J. et al. Huntingtin-associated protein (HAP1): discrete neuronal localizations in the brain resemble those of neuronal nitric oxide synthase. Proc Nat! Acad Sci U S A 93, 4839-4844 (1996). In brief, the modified buffered sucrose solution contained additional 1 mM DTT, 1 mM NaF, 1 mM Na3VO4, 10 nM calyculin-A, 100 nM cyclosporine A. All purification steps were carried out at 4° C. The mouse cerebellum and major blood vessels were removed from the brain samples. Each mouse brain tissue was homogenized in 5 mL of the modified sucrose solution. Cell nuclei and unbroken cells were removed by a 10-minute centrifugation at 800 g. A second centrifugation at 9,200 g for 15 min yielded supernatant (S2) and pellet (P2) fractions. The P2 pellets were resuspended in 10 mL of the modified sucrose solution, followed by another centrifugation at 9,200 g for 15 min to obtain the P2′ fraction. The P2′ fraction was resuspended in the modified sucrose solution, followed by a centrifugation of 10,200 g, 15 min. The resulting pellet, the synaptosomal fraction, was resuspended in a hypotonic solution. Li, X. J. et al. Huntingtin-associated protein (HAP1): discrete neuronal localizations in the brain resemble those of neuronal nitric oxide synthase. Proc Nat! Acad Sci U S A 93, 4839-4844 (1996). The synaptosomal membrane pellet (LP 1) was collected after a centrifugation at 25,000 g for 20 min and resuspended in RIPA buffer (see the Experimental Procedure section) with no protease inhibitors. Trypsin was added to the LP 1 protein solution in a ratio of 50:1 (proteins:trypsin). Trypsin digestion was carried out at 37° C. overnight. Tryptic peptide digests were equally aliquoted to three parts (approximate 100 μg each), one for conventional mass spectrometry, one for neutral-loss of phosphoric acid mass spectrometry, and one for solid-phase chemical tagging mass spectrometry.

### EXAMPLE 4

Cell culture and DNA transfection—Human neuroblastoma CAD cells were cultured in DMEM medium containing 10% fetal bovine serum, 2 mM L-glutamine, 100 units/mL penicillin, and 100 μg/mL streptomycin at 5% CO2, 37° C. At 80-90% confluence, cells were transferred to Opti-MEM media containing glutamine for DNA transfection. Transfection were performed with lipofectamine 2000 following vender's protocol (Invitrogen, Carlsbad, Calif.). Twenty μg of Tau construct, 20 μg of Cdk5 construct, and 20 μg of p25 construct were used for transfection of cells in one 150 cm² flask. Twenty-four hours post transfection, Opti-MEM medium was removed and cells were washed with cold PBS. To generate protein kinase A-Tau, CAD cells were transfected with Tau constructs for 24 hours followed by a 1 hour treatment of 10 μM foskolin (Sigma-Aldorich, St Louis, Mo.).

### EXAMPLE 5

Preparation of Heat-Stable Protein Samples and Immunoblotting Assay

Procedure 1

Tranfected cells were lysed in cold RIPA buffer (150 mM NaCl, 1% NP-40, 1 mM EDTA, 0.5% sodium deoxycholate, 0.1% SDS, 50 mM Tris, pH 8.0) containing 1 mM DTT, 1 mM NaF, 1 mM Na3VO4, 10 nM calyculin-A, 100 mM cyclosporine A. Cell lysates were collected after a short centrifugation to remove cell debris. Tau and MAP2 are heatstable proteins that can be enriched by heat treatment. Grundke-Igbal, I. et al. Microtubule-associated protein tau. A component of Alzheimer paired helical filaments. JBiol Chem 261, 6084-6089 (1986). Cell lysates were heated at 90° C. for 5 min. Heat-stable protein lysates were collected by a 15,000×g centrifugation for 10 min at 4° C. For immunoblotting, 1 μg of each protein sample was resolved on a 12% SDS-PAGE gel. Proteins were electro-transferred onto PVDF membranes. Tau-5 (1:500 dilution), AT-8 (1:1000), PHF-1 (1:500), Tau[pS396] (0.5 μg/ml) were used for the primary antibody binding reaction. HRP-conjugated anti-mouse antibody (1:5000) or HRP-conjugated anti-rabbit antibody (1:5000) was used in secondary antibody binding. The labeled Tau proteins were visualized by chemiluminescence and film exposure.

Procedure 2

Tranfected cells or mouse forebrain tissues were homogenized and lysed in cold RIPA buffer (150 mM NaCl, 1% NP-40, 1 mM EDTA, 0.5% sodium deoxycholate, 0.1% SDS, 50 mM Tris, pH 8.0) containing 1 mM DTT, 1 mM NaF, 1 mM Na₃VO₄, 10 nM calyculin-A, 100 nM cyclosporine A. Cell lysates were collected after short centrifugation to remove cell debris. Tau and MAP2 are heat-stable proteins that can be enriched by heat treatment. Cell lysates were heated at 90° C. for 5 min. Heat-stable protein lysates were collected by a 15,000×g centrifugation for 10 min at 4° C. For immunoblotting, 1 μg of each protein sample was resolved on a 12% SDS-PAGE gel. Proteins were electro-transferred onto PVDF membranes. Tau-5 (1:500 dilution), AT-8 (1:1000), PHF-1 (1:500), and Tau[pS396] (0.5 μg/ml) were used for the primary antibody binding reaction. HRP-conjugated anti-mouse antibody (1:5000) or HRP-conjugated anti-rabbit antibody (1:5000) was used in secondary antibody binding. The labeled Tau proteins were visualized by chemiluminescence and film exposure.

### EXAMPLE 6

Mouse Brain Heat-Stable Phospho-Peptides Enriched by SMA.

Peptides (250 ug) prepared from heat-stable fraction of mouse forebrains were treated with SMA. SMA-resin captured peptides were analyzed by LC-MS/MS. Phosphorylation sites at peptides are labeled in bold fonts. One hundred and five phosphopeptides derived from one 102 proteins are detected. Among total of 183 phosphorylation sites, there are 43 mono-phosphopeptides (41%), 49 di-phospho-peptides (46%), 10 tri-phosphopeptides (10%) and 3 quadru-phospho-peptides (2%). See Table 2.

TABLE 2

| | | | |
|---|---|---|---|
| | | | Number |
| Protein Name | Accession | Detected Peptide Sequence | of Sites |

| | | | | |
|---|---|---|---|---|
| 1 | A kinase anchor protein 1 | O08715 | ₅₄LSEEACPGVLSVAPTVTQPPGR₇₇ | 2 |
| 2 | Acetyl-CoA acetyltransferase | Q8CAY6 | ₉₈AVCLAAQSIAMGDSTIVVAGGMENMSK₁₂₄ | 2 |
| 3 | Acyl coeneyme A:cholesterol | Q8VCC2 | ₂₆₇IAALAGCKTTTSAAMVHCLR₃₈₆ | 2 |
| | acyltransferase | | | |
| 4 | ADAM 2 | Q60718 | ₄₂₅LSDGSECSSGICCNSCK₄₄₂ | 1 |
| 5 | ADAMTS-1 | P97857 | ₇₉₇GTVLRYSGSSAALER₈₁₁ | 2 |
| 6 | ADAMTS-15 | P59384 | ₂₄₇VVDGTLCTPDSTSVCVQGK₂₆₇ | 3 |
| 7 | ADAMT5-20 | P59511 | ₁₆₅₉WSKCSVTCGIGIMER₁₆₇₄ | 2 |
| 8 | ADAMT5-5 | Q9R001 | ₇₂₁CGVCGGDNSSCTKIIGTFNK₇₄₁ | 1 |
| 9 | ATP synthase gamma chain | Q91VR2 | ₂₄₄ESTTSEQSARMTAMDNASK₂₆₂ | 2 |
| 10 | Bcl-2-associated transcription | Q8K019 | ₁₄₁SSSSSASPSSPSSR₁₅₄ | 1 |
| | factor 1 | | | |
| 11 | Brain link protein 2 | Q80WM4 | ₂₂₉EPCGGTGSTGAGGGTNGGVR₂₄₈ | 1 |
| 12 | BRCA1-associated RING domain | O70445 | ₄₀₃LSSGMPAR₄₁₀ | 2 |
| | protein 1 | | | |
| 13 | Breast cancer type 2 susceptibility | P97929 | ₂₂₀₁CSLFTCPQNETLFNSRTR₂₂₁₈ | 2 |
| | protein homolog | | | |
| 14 | Carboxypeptidase D | O89001 | ₈₈₅GHSTSTDDTSDPTSKEFEALIK₉₀₆ | 3 |
| 15 | CBL E3 ubiguitin protein ligase | P22882 | ₈SSGAGGGGSGGSGAGGLMK₂₉ | 2 |
| 16 | CCR4-NOT transcription complex | Q8C5L3 | ₁₆₉SSPSIICMPK₁₇₈ | 2 |
| | subunit 2 | | | |
| 17 | Cell division cycle 7-related | Q9ZOHO | ₄₅₈GLDSTTPR₄₆₅ | 1 |
| | protein kinase | | | |
| 18 | Coatomer gamma-2 subunit | Q9QXK3 | ₁₅₂VSSVASSALVSSLHMMK₆₈ | 3 |
| 19 | CRK-associated substrate | Q61140 | ₆₁₂TKAPGPGPEGSSSLHPNPTDK₆₃₂ | 2 |
| 20 | Cyctin-dependent kinase 5 activator | O35926 | ₃₂₇NEGEAAASTGGPPSGSSASTTSSSSAR₃₅₃ | 3 |
| | 2, p39 | | | |
| 21 | Cytokine receptor-like factor 2 | Q8C119 | ₃₃₅GPGGGAMVSVGGATFMVGDSGYMTL₃₅₉ | 2 |
| 22 | Desmoglein 2 | O55111 | ₇₀₄GSSSASVTK₇₁₂ | 1 |
| 23 | Disabled homolog 2 | P98078 | ₃₉₃SSPNPFVGSPPKGLSVPNGVK₄₁₃ | 3 |
| 24 | DNA-binding protein SATB | Q8V124 | ₄₄₀SMNPNVSMVSSASSSPSSSRTPQAK₄₆₄ | 2 |
| 25 | Ephrin type-A receptor 1 | Q60750 | ₁₀₈DCKSFPGGAGPLGCK₁₂₂ | 1 |
| 26 | Ephrin type-B recepior 3 | P54754 | ₃₇₂CRGSSGAGGPATCSR₃₈₆ | 2 |
| 27 | Epidermis-type lipoxygenase 3 | Q9WV07 | ₁₆AGTLDNIYATLVGTCGESPK₃₅ | 1 |
| 28 | ETS domain transcription factor ERP | P70459 | ₅₁₇GDVGPGESGGPLTPRR₅₃₂ | 1 |
| 29 | F-box/LRR-repeat protein 8 | Q8C1G9 | ₃₄AWAAAAANSTVWSDK₄₈ | 2 |
| 30 | Formic 2 | Q9JL04 | ₃₆₅ESITSAVASLPGSPAPSPR₃₈₃ | 1 |
| 31 | Gamma-aminobutyric-acid receptor | P16305 | ₃₉₉STPVSPPLLLPATGGTSK₄₁₆ | 2 |
| | alpha-6 subunit | | | |
| 32 | Glutamate receptor 2 | P23819 | ₇₉₂GECGSGGGDSK₈₀₂ | 1 |
| 33 | Guanine nucleotide-binding protein | Q9CXP8 | ₂SSGASVSALQR₁₂ | 2 |
| | G(I)/G(S)/G(O) gamma-10 subunit | | | |
| 34 | Guanine nucleotide-binding protein G(o), | P18873 | ₁GCTLSAEERAALER₁₄ | 1 |
| | alpha subunit 2 | | | |
| 35 | Hairiess protein | Q61645 | ₃₄₂CQDSPEGGSSGPGESSEERNK₃₆₂ | 3 |
| 36 | Heterogeneous nuclear ribonucleoprotein A1 | P49312 | ₃₀₀NQGGYGGSSSSSSYGSGRR₃₁₈ | 1 |
| | | | ₃₀₀NQGGYGGSSSSSSYGSGR₃₁₇ | 3 |
| 37 | High-affinity cAMP-specific 3,5′-cyclic | P70453 | ₄₁₀SQTMLGHVGLNK₄₂₂ | 2 |
| | phosphodiesterase 7A | | | |
| 38 | Histone-lysine N-methyltransferase | Q9Z148 | ₁₆₈SFPSSPSKGGACPSR₁₈₂ | 2 |
| 39 | Homeobos protein SIX3 | Q6233 | ₅₄AGGGGAGGAGGGSGGGGSR₇₄ | 1 |

### EXAMPLE 7

Preparation of Peptides and IMAC Sample

Phospho-Thr VDAAV(phospho-T)PEERHC peptides (1 mg/mL) were incubated with iodoacetamide (10 mg/mL) for 30 min at room temperature in dark. The carbamodimethylation was quenched by addition of 8 mM DTT. Recombinant Tau was prepared as described previously[14]. The in vitro phosphorylation conditions of Tau (1 μg) was previously described[14], except that 50 ng of purified CDK5/p25 was used instead. The in vitro phosphorylation of MAP2 used the same condition as that of Tau. Phosphorylated Tau was digested with trypsin at a ratio of 100:1 (w/w) at 37° C. overnight. Phosphorylation was stopped by adding acetic acid when Tau was phosphorylated to 0.4-0.5 phosphates per Tau molecule. Dephosphorylation of CDK5-Tau peptides was performed with 5 units of alkaline phosphatase at 37° C. for four hours. The IMAC procedure in this study followed the vender instructions. Phos-Select iron affinity gel (100 μL) was incubated with Tau phospho-peptides. The binding condition of Tau peptides and IMAC resin was 250 mM acetic acid in 25% acetonitrile. Bound peptides were eluted in 150 mM ammonuion hydroxide in 25% acetonitrile. In microtubule-associated protein experiments, 1 mg of heat-stable proteins were treated with sequencing-grade trypsin at a ratio of 50:1 (proteins: trypsin) in 50 mM ammonium bicarbonate at 37° C. for 16 hours. All peptides (synthetic and expressed) were transferred to PlusOne Mini dialysis bags with 1 kDa cut-off (Amersham Bioscience, Piscataway, N.J.) and dialyzed against water at 4° C. overnight. A C18 reverse phase chromatography may be used to substitute the dialysis step. Lyophilized peptides were stored at −20° C. for the subsequent capture-release-tag procedure and mass spectrometry.

In MAP experiments, heat-stable proteins were treated with sequencing-grade trypsin at a ratio of 50:1 (proteins: trypsin) at 37° C. for 16 hours. All peptides (synthetic and expressed) were transferred to a PlusOne Mini dialysis bag with 1 kDa cut-off (Amersham Bioscience, Piscataway, N.J.) and dialyzed against water at 4° C. overnight. Lyophilized peptides were stored at −20° C. for the subsequent capturerelease-tag procedure and mass spectrometric assay.

### Incorporation by Reference

All of the U.S. patents and U.S. patent application publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

1. A method of labeling a peptide or protein comprising a phosphorylated amino acid comprising:
a) removing from the peptide or protein the phosphate group via a β-elimination to form a double bond conjugated to a C═O group;
b) reacting the double bond from step a) with a nucleophile comprising a tag and a solid support; and
c) removing the solid support from the product of step b) forming the labeled peptide or protein.

2. The method of claim 1, wherein removing the phosphate group via a β-elimination is base catalyzed.

3. The method of claim 1, wherein removing the phosphate group via β-elimination is catalyzed by an amine-containing base, a hydroxide-containing base, or mixtures thereof.

4. The method of claim 1, wherein removing the phosphate group via β-elimination is catalyzed by a hydroxide-containing base.

5. The method of claim 1, wherein removing the phosphate group via β-elimination is catalyzed by Ba(OH)₂.

6. The method of claim 1, wherein the nucleophile is selected from the group consisting of oxygen-containing nucleophiles, nitrogen-containing nucleophiles, and sulfur-containing nucleophiles.

7. The method of claim 1, wherein the nucleophile comprises a sulfur atom.

8. The method of claim 1, wherein one or more other potential nucleophiles within the peptide or protein are protected before step a).

9. The method of claim 1, wherein one or more other potential nucleophiles within the peptide or protein are protected before step a) via acylation or alkylation.

10. The method of claim 1, wherein the tag comprises a deuterium ion.

11. The method of claim 1, wherein the tag comprises eight deuterium ions.

12. The method of claim 1, wherein the solid support is a glass, derivatized glass, silicon, plastic or a resin.

13. The method of claim 1, wherein the solid support is a bead.

14. The method of claim 1, wherein the solid support is a resin bead.

15. The method of claim 1, wherein the solid support is a polystyrene/poly(ethylene glycol) resin bead.

16. The method of claim 1, wherein the solid support is removed by acid hydrolysis.

17. The method of claim 1, wherein the efficiency in detecting the peptides and proteins is increased by about 60% as compared to detection by mass spectrometry.

18. The method of claim 1, wherein there is a linker between said solid support and said nucleophile-containing tag.

19. The method of claim 18, wherein the linker is a NH₂-functionalized RINK linker (NH₂-RINK linker), 4-hydroxymethyl-phenylacetamidomethyl linker (PAM linker), 4-(1′,1′-dimethyl-1′-hydroxypropyl)phenoxyacetyl linker (DHPP linker), 4-hydroxymethylphenoxy linker (Wang linker), super Acid Sensitive linker (SASRIN linker), hypersensitive acid-labile tris(alkoxy)benzyl ester linker (HAL linker), 4-(2′,4′-dimethoxyphenyl-Fmoc-aminomethyl)-phenoxy linker (RINK linker), N-[9-(Hydroxymethyl)-2-fluorenyl]succinamic acid linker (HMFS linker), fmoc-(aminomethyl)-3,5-dimethoxyvaleric acid linker (Pal linker), 9-xanthenyl linker (XAN linker) or safety-catch acid-labile linker (SCAL linker).

20. The method of claim 18, wherein the linker is a NH₂-functionalized RINK linker (NH₂-RINK linker) or 4-(2′,4′-dimethoxyphenyl-Fmoc-aminomethyl)-phenoxy linker (RINK linker).

21. The method of claim 18, wherein the linker is a NH₂-functionalized RINK linker (NH₂-RINK linker).

22. The method of claim 18, wherein the solid support is a polystyrene/poly(ethylene glycol) resin bead; the linker is a NH₂-functionalized RINK linker (NH₂-RINK linker); the tag comprises a deuterium ion; and the nucleophile comprises a sulfur atom.

23. The method of claim 18, wherein the solid support is a polystyrene/poly(ethylene glycol) resin bead; the linker is a NH₂-functionalized RINK linker (NH₂-RINK linker); the tag and nucleophile is —C(═O)CD₂CD₂CD₂CD₂CH₂NHC(═O)CH₂SX; and X is a cation.

24. A solid support comprising a linker, a tag and a nucleophile.

25. The solid support of claim 24, wherein the solid support is a glass, derivatized glass, silicon, plastic or a resin.

26. The solid support of claim 24, wherein the solid support is a bead.

27. The solid support of claim 24, wherein the solid support is a resin bead.

28. The solid support of claim 24, wherein the solid support is a polystyrene/poly(ethylene glycol) resin bead.

29. The solid support of claim 24, wherein the tag contains a deuterium label.

30. The solid support of claim 24, wherein the tag contains at least eight deuterium atoms.

31. The solid support of claim 24, wherein the linker comprises an amide moiety.

32. The solid support of claim 24, wherein the linker comprises an aryl moiety.

33. The solid support of claim 24, wherein the linker is a NH₂-functionalized RINK linker (NH₂-RINK linker), 4-hydroxymethyl-phenylacetamidomethyl linker (PAM linker), 4-(1′,1′-dimethyl-1′-hydroxypropyl)phenoxyacetyl linker (DHPP linker), 4-hydroxymethylphenoxy linker (Wang linker), super Acid Sensitive linker (SASRIN linker), hypersensitive acid-labile tris(alkoxy)benzyl ester linker (HAL linker), 4-(2′,4′-dimethoxyphenyl-Fmoc-aminomethyl)-phenoxy linker (RINK linker), N-[9-(Hydroxymethyl)-2-fluorenyl]succinamic acid linker (HMFS linker), fmoc-(aminomethyl)-3,5-dimethoxyvaleric acid linker (Pal linker), 9-xanthenyl linker (XAN linker) or safety-catch acid-labile linker (SCAL linker).

34. The solid support of claim 24, wherein the linker is a NH₂-functionalized RINK linker (NH₂-RINK linker) or 4-(2′,4′-dimethoxyphenyl-Fmoc-aminomethyl)-phenoxy linker (RINK linker).

35. The solid support of claim 24, wherein the linker is a NH₂-functionalized RINK linker (NH₂-RINK linker).

36. The solid support of claim 24, wherein the nucleophile is selected from the group consisting of oxygen-containing nucleophiles, nitrogen-containing nucleophiles, and sulfur-containing nucleophiles.

37. The solid support of claim 24, wherein the nucleophile comprises a sulfur atom.

38. The solid support of claim 24, wherein the solid support is a polystyrene/poly(ethylene glycol) resin bead; the linker is a NH₂-functionalized RINK linker (NH₂-RINK linker); the tag comprises a deuterium ion; and the nucleophile comprises a sulfur atom.

39. The solid support of claim 24, wherein the solid support is a polystyrene/poly(ethylene glycol) resin bead; the linker is a NH₂-functionalized RINK linker (NH₂-RINK linker); the tag and nucleophile is —C(═O)CD₂CD₂CD₂CD₂CH₂NHC(═O)CH₂SX; and X is a cation.
